(19) United States
(12) Patent Application Publication
(10) Pub. No.: US 2016/0067040 A1
(43) Pub. Date: Mar. 10, 2016

(54) VALVE LOCKING MECHANISM

(71) Applicant: BOSTON SCIENTIFIC SCIMED, INC., MAPLE GROVE, MN (US)

(72) Inventors: Sumit Agrawal, Woodbury, MN (US); Nawaz Maditheti, Jacksonville, FL (US); Eric M. Petersen, Maple Grove, MN (US); Stephen J. Oommen, St. Paul, MN (US); James A. Schmidt, Monticello, MN (US)

(73) Assignee: BOSTON SCIENTIFIC SCIMED, INC., MAPLE GROVE, MN (US)

(21) Appl. No.: 14/845,824

(22) Filed: Sep. 4, 2015

Related U.S. Application Data
(60) Provisional application No. 62/047,914, filed on Sep. 9, 2014.

(51) Int. Cl. A61F 2/24

(52) U.S. Cl. CPC A61F 2/2427

(57) ABSTRACT

A medical device may include an elongate delivery sheath and a valve replacement implant disposed within a lumen of the delivery sheath, the implant including an anchor member reversibly actuatable between a delivery configuration and a deployed configuration. The implant may include at least one locking element configured to lock the anchor member in the deployed configuration, and at least one actuator element configured to engage the at least one locking element and actuate the anchor member between the delivery configuration and the deployed configuration. The at least one actuator element may include an unlocking member configured to compress a first locking portion of the at least one locking element to unlock the anchor member from the deployed configuration.

### CROSS-REFERENCE TO RELATED APPLICATIONS

This application claims priority to U.S. Provisional Application No. 62/047,914, filed Sep. 9, 2014.

### TECHNICAL FIELD

The present disclosure pertains to medical devices, and methods for manufacturing and/or using medical devices. More particularly, the present disclosure pertains to locking mechanisms for a replacement heart valve.

### BACKGROUND

A wide variety of intracorporeal medical devices have been developed for medical use, for example, intravascular use. Some of these devices include guidewires, catheters, medical device delivery systems (e.g., for stents, grafts, replacement valves, etc.), and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

In a first aspect, a medical device may include an elongate delivery sheath, and a valve replacement implant disposed within a lumen of the delivery sheath, the implant including an anchor member reversibly actuatable between a delivery configuration and a deployed configuration. The implant may include at least one locking element configured to lock the anchor member in the deployed configuration, and at least one actuator element configured to engage the at least one locking element and actuate the anchor member between the delivery configuration and the deployed configuration. The at least one actuator element may include an unlocking member configured to compress a first locking portion of the at least one locking element, thereby unlocking the anchor member from the deployed configuration.

In addition or alternatively, and in a second aspect, the first locking portion of the at least one locking element is fixedly attached to the anchor member and a second locking portion of the at least one locking element is fixedly attached to the anchor member, the first locking portion and the second locking portion being longitudinally movable relative to each other in the delivery configuration.

In addition or alternatively, and in a third aspect, the first locking portion is fixedly attached to a distal portion of the anchor member.

In addition or alternatively, and in a fourth aspect, the second locking portion is fixedly attached to a proximal portion of the anchor member.

In addition or alternatively, and in a fifth aspect, the first locking portion engages the second locking portion in the deployed configuration.

In addition or alternatively, and in a sixth aspect, the second locking portion includes a circumferential frame defining a passageway and the first locking portion includes an elongated member having a passage therethrough, wherein the passageway is configured to receive the elongated member therein.

In addition or alternatively, and in a seventh aspect, the tubular member includes a tapered end portion configured to engage the circumferential frame when the elongated member is translated through the passageway.

In addition or alternatively, and in an eighth aspect, the tapered end portion includes a first end and a second end, the tapered end portion being tapered from a first outer extent at the first end to a second outer extent at the second end, wherein the second outer extent is less than the first outer extent, and the first end defines a shoulder of the elongated member oriented generally transverse to a longitudinal axis of the elongated member.

In addition or alternatively, and in a ninth aspect, the tapered end portion is configured to flex inward as the tapered end portion is translated through the passageway.

In addition or alternatively, and in a tenth aspect, the circumferential frame includes at least one recess extending outward from the passageway, the at least one recess being configured to engage the shoulder.

In addition or alternatively, and in an eleventh aspect, translating the second end of the tapered end portion through the passageway until the shoulder has engaged the at least one recess locks the anchor member in the deployed configuration.

In addition or alternatively, and in a twelfth aspect, the circumferential frame includes at least one projection extending inwardly into the passageway, the at least one projection being configured to engage the shoulder.

In addition or alternatively, and in a thirteenth aspect, the at least one actuator element extends through the second locking portion and is releasably coupled to the first locking portion, the at least one actuator element being axially translatable through the second locking portion.

In addition or alternatively, and in a fourteenth aspect, the unlocking member is axially translatable through the second locking portion.

In addition or alternatively, and in a fifteenth aspect, the unlocking member includes a cavity disposed therein, the cavity being configured to receive at least a portion of the first locking portion.

In addition or alternatively, and in a sixteenth aspect, axial translation of the at least one actuator element in a first direction actuates the anchor member from the delivery configuration to the deployed configuration.

In addition or alternatively, and in a seventeenth aspect, axial translation of the at least one actuator element in a second direction opposite from the first direction engages the unlocking member with the tapered end portion to unlock the anchor member from the deployed configuration.

In addition or alternatively, and in an eighteenth aspect, further axial translation of the at least one actuator element in the second direction actuates the anchor member from the deployed configuration to the delivery configuration.

In addition or alternatively, and in a nineteenth aspect, a medical device may include an elongate delivery sheath, and a medical implant disposed within a lumen of the delivery sheath and attached to an inner catheter, the implant including an anchor member reversibly actuatable between an elongated delivery configuration and an expanded deployed configuration. The implant may include first, second, and third locking elements configured to lock the anchor member in the deployed configuration, and first, second, and third actuator elements configured to engage the first, second, and third locking elements and actuate the anchor member between the delivery configuration and the deployed configuration. The first, second, and third actuator elements may each include an unlocking member having a cavity configured to receive and compress a first locking portion of each of the first, second, and third locking elements to unlock the anchor member from the deployed configuration.

In addition or alternatively, and in a twentieth aspect, distal axial translation of the first, second, and third actuator elements may compress the first locking portion of each of the first, second, and third locking elements laterally inward, thereby disengaging the first locking portion from a second locking portion of the locking elements fixedly attached to the anchor member.

The above summary of some embodiments, aspects, and/or examples is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic side view of an example medical device system;

FIG. 2 is a perspective view of a portion of an example implant associated with the example medical device system in a deployed configuration;

FIG. 3 illustrates selected components of an example implant associated with the example medical device system in a delivery configuration;

FIG. 4 is a cross-sectional view taken through line 4-4 in FIG. 3;

FIG. 5 is a cross-sectional view of the selected components illustrated in FIG. 4 partially translated toward a deployed configuration;

FIG. 6 illustrates selected components of an example implant associated with the example medical device system in a deployed configuration;

FIG. 7 is a cross-sectional view taken through line 7-7 in FIG. 6;

FIG. 8 illustrates selected components of an example implant associated with the example medical device system partially translated from the deployed configuration toward the delivery configuration;

FIG. 9 is a cross-sectional view taken through line 9-9 in FIG. 8;

FIG. 10 illustrates selected components of an example implant associated with the example medical device system further translated from the deployed configuration toward the delivery configuration;

FIG. 11 is a cross-sectional view taken through line 11-11 in FIG. 10;

FIG. 12 illustrates selected components of an example implant associated with the example medical device system further translated from the deployed configuration toward a released configuration;

FIG. 13 illustrates selected components of an example implant associated with the example medical device system further translated from the deployed configuration toward a released configuration;

FIG. 14 illustrates selected components of an example implant associated with the example medical device system in the released configuration;

FIGS. 15-20 illustrate selected components of an example implant associated with an example medical device system;

FIG. 21 is a partial cross-sectional view taken through line 21-21 in FIG. 20;

FIGS. 22-23 illustrate selected components of an example implant associated with an example medical device system;

FIG. 24 is a partial cross-sectional view taken through line 24-24 in FIG. 6;

FIG. 25 is a partially exploded view of selected components of an example implant associated with an example medical device system;

FIG. 26 illustrates selected components of the example implant associated with the example medical device system of FIG. 25;

FIG. 27 is a partial cross-sectional view taken through line 27-27 in FIG. 26;

FIG. 28 illustrates selected components of the example implant associated with the example medical device system of FIG. 25; and

FIG. 29 is a partial cross-sectional view taken through line 29-29 in FIG. 28.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the claimed invention. Those skilled in the art will recognize that the various elements described and/or shown may be arranged in various combinations and configurations without departing from the scope of the disclosure. The detailed description and drawings illustrate example embodiments of the claimed invention.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (i.e., having the same function or result). In many instances, the term “about” may include numbers that are rounded to the nearest significant figure. Other uses of the term “about” (i.e., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect such feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangable with each other to form other additional embodiments or to complement and/or enrich the described embodiment(s), as would be understood by one of ordinary skill in the art.

Diseases and/or medical conditions that impact the cardiovascular system are prevalent in the United States and throughout the world. Traditionally, treatment of the cardiovascular system was often conducted by directly accessing the impacted part of the system. For example, treatment of a blockage in one or more of the coronary arteries was traditionally treated using coronary artery bypass surgery. As can be readily appreciated, such therapies are rather invasive to the patient and require significant recovery times and/or treatments. More recently, less invasive therapies have been developed, for example, where a blocked coronary artery could be accessed and treated via a percutaneous catheter (e.g., angioplasty). Such therapies have gained wide acceptance among patients and clinicians.

Some relatively common medical conditions may include or be the result of inefficiency, ineffectiveness, or complete failure of one or more of the valves within the heart. For example, failure of the aortic valve can have a serious effect on a human and could lead to serious health condition and/or death if not dealt with. Treatment of defective heart valves poses other challenges in that the treatment often requires the repair or outright replacement of the defective valve. Such therapies may be highly invasive to the patient. Disclosed herein are medical devices that may be used for delivering a medical device to a portion of the cardiovascular system in order to diagnose, treat, and/or repair the system. At least some of the medical devices disclosed herein may be used to deliver and implant a replacement heart valve (e.g., a replacement aortic valve). In addition, the devices disclosed herein may deliver the replacement heart valve percutaneously and, thus, may be much less invasive to the patient. The devices disclosed herein may also provide a number of additional desirable features and benefits as described in more detail below.

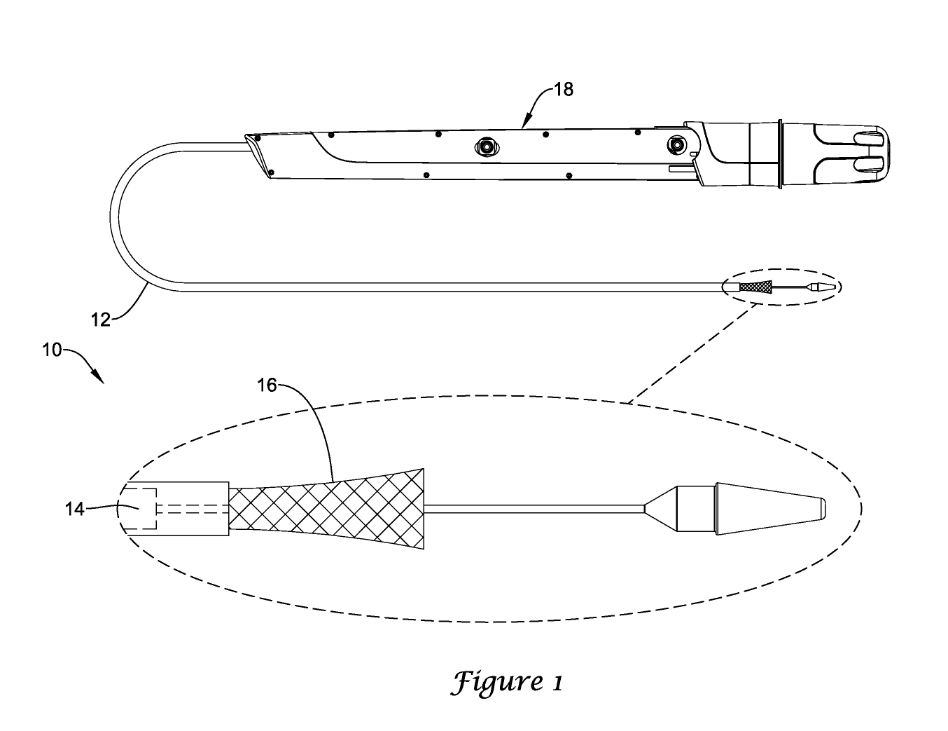

FIG. 1 is a side view of an example medical device system 10. It should be noted that some features of the medical device system 10 are either not shown, or are shown schematically, in FIG. 1 for simplicity. Additional details regarding some of the components of the medical device system 10 are provided in other figures in greater detail. A medical device system 10 may be used to deliver and/or deploy a variety of medical devices to a number of locations within the anatomy. In at least some embodiments, the medical device system 10 may be a replacement heart valve delivery system (e.g., a replacement aortic valve delivery system) that can be used for percutaneous delivery of a replacement heart valve. This, however, is not intended to be limiting as the medical device system 10 may also be used for other interventions including mitral valve replacement, valve repair, valvuloplasty, and the like, or other similar interventions.

The medical device system 10 may generally be described as a catheter system that includes a catheter or an outer sheath 12 and a tube or an inner catheter 14 (a portion of which is shown in FIG. 1 in phantom line) extending at least partially through the outer sheath 12. A medical implant 16 (i.e., a valve replacement implant, for example) may be coupled to the inner catheter 14 and disposed within a lumen of the outer sheath 12 during delivery of the medical implant 16. A handle 18 may be disposed at a proximal end of the outer sheath 12 and/or the inner catheter 14. In general, the handle 18 may be configured to manipulate the position of the outer sheath 12 relative to the inner catheter 14, as well as aid in the deployment of the medical implant 16.

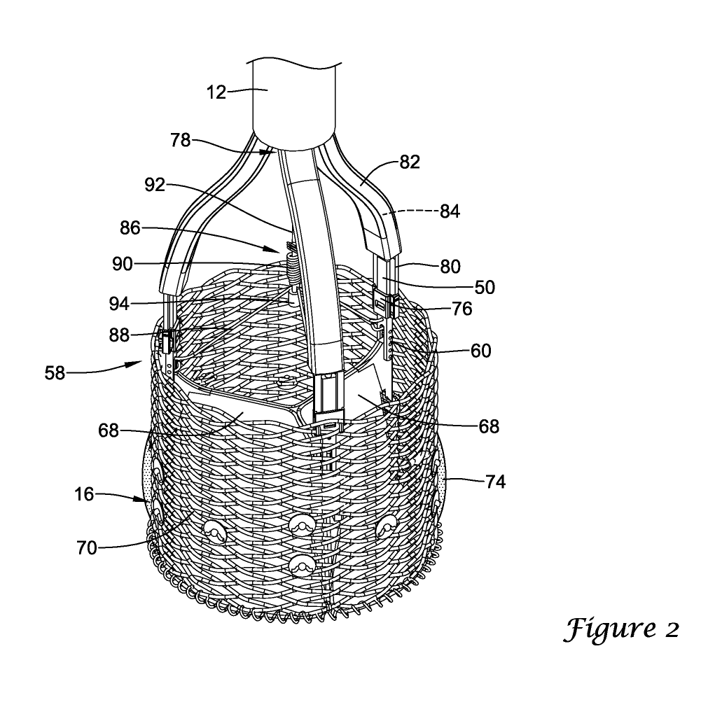

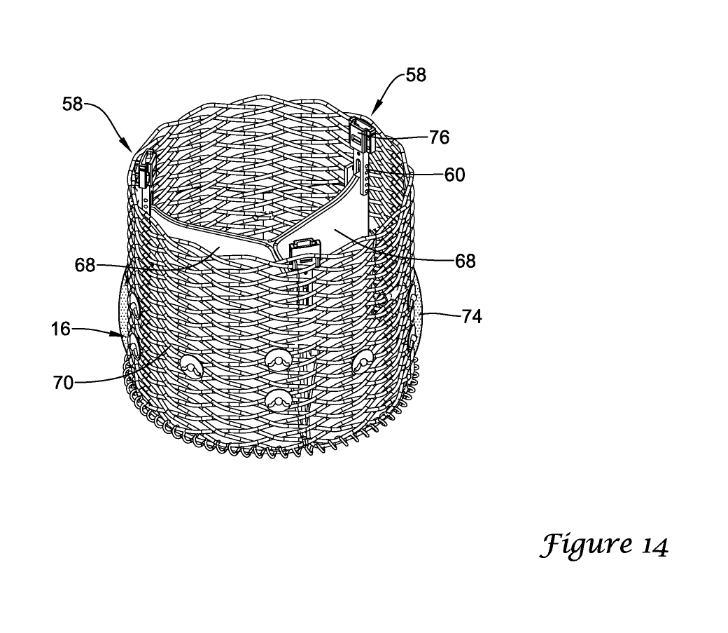

In use, the medical device system 10 may be advanced percutaneously through the vasculature to a position adjacent to an area of interest. For example, the medical device system 10 may be advanced through the vasculature to a position adjacent to a defective aortic valve. During delivery, the medical implant 16 may be generally disposed in an elongated and low profile “delivery” configuration within the outer sheath 12 (as partially shown in FIG. 1, for example). Once positioned, the outer sheath 12 may be retracted to expose the medical implant 16. The medical implant 16 may be actuated in order to radially expand the medical implant 16 into a generally shortened and larger profile “deployed” configuration suitable for implantation within the anatomy (as shown in FIG. 2, for example). When the medical implant 16 is suitably deployed within the anatomy, the medical device system 10 can be removed from the vasculature, leaving the medical implant 16 in place in a “released” configuration to function as, for example, a suitable replacement for the native aortic valve (as seen in FIG. 14, for example). In at least some interventions, the medical implant 16 may be deployed within the native valve (e.g., the native valve is left in place and not excised). Alternatively, the native valve may be removed and the medical implant 16 may be deployed in its place as a replacement.

In some embodiments, the outer sheath 12 may have a proximal portion and a distal portion. In some embodiments, the distal portion may have a slightly enlarged or flared inner diameter, which may provide additional space for holding the medical implant 16 therein. For example, in some embodiments, an inner diameter of outer sheath 12 along a proximal portion may be in the range of about 0.254 to 1.27 cm (0.10 to 0.50 inches), or about 0.508 to 1.016 cm (0.20 to 0.40 inches), or about 0.508 to 0.762 cm (0.20 to 0.30 inches), or about 0.56388±0.0508 cm (0.222±0.002 inches). In some embodiments, an inner diameter of outer sheath 12 along a distal portion may be in the range of about 0.254 to 1.27 cm (0.10 to 0.50 inches), or about 0.508 to 1.016 cm (0.20 to 0.40 inches), or about 0.508 to 0.762 cm (0.20 to 0.30 inches), or about 0.579 to 0.5842 cm (0.228 to 0.230 inches). At the distal end of the distal portion may be a distal tip, which may be flared or otherwise have a funnel-like shape. The funnel-like shape may increase the outer diameter (and inner diameter) of the outer sheath 12 at the distal tip and may aid in the sheathing and/or re-sheathing of the medical implant 16 into the outer sheath 12. Other than at the distal tip, the outer sheath 12 may have a generally constant outer diameter. For example, in some embodiments, the outer sheath 12 may have an outer diameter in a range of about 0.254 to 1.27 cm (0.10 to 0.50 inches), or about 0.508 to 1.016 cm (0.20 to 0.40 inches), or about 0.508 to 0.762 cm (0.20 to 0.30 inches), or about 0.6858 cm (0.270 inches). These are just examples. Other embodiments are contemplated that have differing dimensions (including those appropriate for differently sized patients including, but not limited to, children) and/or arrangements for the outer diameter and/or inner diameter of the outer sheath 12. These contemplated embodiments include outer sheaths with flared or otherwise variable outer diameters, embodiments with constant inner diameters, combinations thereof, and the like. The outer sheath 12 may also have a length that is appropriate for reaching the intended area of interest within the anatomy. For example, the outer sheath 12 may have a length in the range of about 30 to 200 cm, or about 60 to 150 cm, or about 100 to 120 cm, or about 108±0.20 cm. In some embodiments, some, all, or a portion of the outer sheath 12 may also be curved. For example, in some embodiments, a distal section of outer sheath 12 may be curved. In one example, a radius of the curve (measured from the center of outer sheath 12) may be in the range of about 2 to 6 cm (20 to 60 mm), or about 3 to 4 cm (30 to 40 mm), or about 3.675 cm (36.75 mm). Again, these dimensions are examples and are not intended to be limiting.

In some embodiments, the outer sheath 12 may be formed from a singular monolithic tube or unitary member. Alternatively, the outer sheath 12 may include a plurality of layers or portions. In some embodiments, one or more of these layers may include a reinforcing structure such as a braid, coil, mesh, combinations thereof, or the like. In some embodiments, a reinforcement or reinforcement layer may be disposed on an intermediate layer. In some embodiments, an outer coating (e.g., a lubricious coating, a hydrophilic coating, a hydrophobic coating, etc.) may be disposed along portions or all of an outer layer. These are just examples. Other alternative structural configurations are also contemplated.

The dimensions and materials utilized for the various layers of the outer sheath 12 may also vary. For example, an inner layer may include a polymeric material such as fluorinated ethylene propylene (FEP) and may have a thickness in the range of about 0.00254 to 0.0127 cm (0.001 to 0.005 inches) or about 0.00762±0.00254 (0.003±0.001 inches), an intermediate layer may include a polymer material such as polyether block amide (e.g., PEBAX 6333) and may have a thickness in the range of about 0.00254 to 0.0127 cm (0.001 to 0.005 inches) or about 0.00508±0.00254 (0.002±0.001 inches), an outer layer may include a polymer material such as polyether block amide (e.g., PEBAX 7233) and may have a thickness in the range of about 0.00254 to 0.0254 cm (0.001 to 0.01 inches). In some embodiments, the outer layer may vary in thickness. For example, along the proximal portion, the outer layer may have greater thickness, such as about 0.0127 to about 0.0508 cm or about 0.02159 cm (0.005 to 0.02 inches or about 0.0085 inches), than along the distal portion and/or at the distal tip, which may be about 0.0127 to about 0.0508 cm or about 0.01651 cm (e.g., about 0.005 to 0.02 inches or about 0.0065 inches). These are just examples as other suitable materials may be used.

The form of the distal tip may also vary. For example, in at least some embodiments, the inner liner layer (i.e., a 2.5 mm section thereof, for example) may be extended up and around the distal end of the outer sheath 12. In some embodiments, a ring member (not shown) made from a suitable material such as a 55D polyether block amide (e.g., 55D PEBAX) may be disposed over the inner layer and heat bonded to form the distal tip. In some embodiments, this may form the funnel-like shape of the distal tip.

In some embodiments, a reinforcement or reinforcement layer may take the form of a braid, coil, mesh, or the like. For example, in some embodiments, the reinforcement or reinforcement layer may include a metallic braid (e.g., stainless steel). In some of these embodiments, the reinforcement or reinforcement layer may also include additional structures such as one or more longitudinally-extending strands. For example, the reinforcement or reinforcement layer may include a pair of longitudinally-extending aramid and/or para aramid strands (for example, KEVLAR®) disposed on opposite sides of the braid. These strands may or may not be woven into portions or all of the braid.

In some embodiments, a distal end region of the inner catheter 14 may include a stepped outer diameter that defines a decreased outer diameter section. For example, the decreased outer diameter section may have an outer diameter in a range of about 0.127 to 0.635 cm (0.05 to 0.25 inches), or about 0.254 to 0.508 cm (0.10 to 0.20 inches), or about 0.38608±0.00762 (0.152±0.003 inches) as opposed to the remainder of the inner catheter 14 where the outer diameter may be in a range of about 0.127 to 0.762 cm (0.05 to 0.30 inches), or about 0.254 to 0.635 cm (0.10 to 0.25 inches), or about 0.508±0.0254 cm (0.20±0.01 inches). In some embodiments, the decreased outer diameter section may define a region where other components of the medical device system 10 may be attached.

In general, the inner catheter 14 may take the form of an extruded polymer tube. Other forms are also contemplated including other polymer tubes, metallic tubes, reinforced tubes, or the like including other suitable materials such as those disclosed herein. In some embodiments, the inner catheter 14 is a singular monolithic or unitary member. In other embodiments, the inner catheter 14 may include a plurality of portions or segments that are coupled together. A total length of the inner catheter 14 may be in a range of about 60 to 150 cm, or about 80 to 120 cm, or about 100 to 115 cm, or about 112±0.02 cm. Just like the outer sheath 12, in some embodiments, the inner catheter 14 may also be curved, for example adjacent to a distal end thereof. In some embodiments, the inner catheter 14 may have one or more sections or regions with a differing hardness/stiffness (e.g., differing shore durometer). For example, in some embodiments, the inner catheter 14 may have a proximal region and an intermediate region. The proximal region may include a generally stiff polymeric material such as a 72D polyether block amide (e.g., 72D PEBAX) and may have a length in a range of about 60 to 150 cm, or about 80 to 120 cm, or about 100 to 115 cm, or about 109.5±0.02 cm. The intermediate region may include a 40D polyether block amide (e.g., 40D PEBAX) and may have a length in a range of about 5 to 25 mm, or about 10 to 20 mm, or about 15±0.01 mm. In some embodiments, the decreased outer diameter section may also differ from the proximal region and/or the intermediate region, and in some embodiments, may include a 72D polyether block amide (e.g., 72D PEBAX) and may have a length in the range of about 0.5 to 2 cm (5 to 20 mm), or about 0.8 to 1.5 cm (8 to 15 mm), or about 1±0.001 cm (10±0.01 mm). These are just examples.

In some embodiments, the inner catheter 14 may include one or more lumens extending therethrough. For example, in some embodiments, the inner catheter 14 may include a first lumen, a second lumen, a third lumen, and a fourth lumen. In general, the one or more lumens extend along an entire length of the inner catheter 14. Other embodiments are contemplated, however, where one or more of the one or more lumens extend along only a portion of the length of the inner catheter 14. For example, in some embodiments, the fourth lumen may stop just short of a distal end of the inner catheter 14 and/or be filled in at its distal end to effectively end the fourth lumen proximal of the distal end of the inner catheter 14.

Disposed within a first lumen of the inner catheter 14 may be at least one actuator element, such as a push-pull rod 84 for example, which may be used to actuate (i.e., expand and/or elongate) the medical implant 16 between a delivery configuration and a deployed configuration. In some cases, the push-pull rod(s) 84 may herein be referred to, or used interchangeably with, the term “actuator element”. In other words, the medical device system 10 may include at least one push-pull rod 84. In some embodiments, the at least one push-pull rod 84 may include two push-pull rods 84, three push-pull rods 84, four push-pull rods 84, or another suitable or desired number of push-pull rods 84. For the purpose of illustration only, the medical device system 10 and/or the medical implant 16 is shown with three push-pull rods 84.

In at least some embodiments, the first lumen may be lined with a low friction liner (e.g., a FEP liner). Disposed within a second lumen may be a pin release mandrel 92, which is explained in more detail herein. In at least some embodiments, the second lumen may be lined with a hypotube liner. A third lumen may be a guidewire lumen and in some embodiments, the third lumen may also be lined with a hypotube liner. In some embodiments, a fourth lumen may be used to house a non-stretch wire. The form of non-stretch wire may vary. In some embodiments, the non-stretch wire may take the form of a stainless steel braid. The non-stretch wire may optionally include a pair of longitudinally-extending aramid and/or para aramid strands (for example, KEVLAR®) disposed on opposite sides of the braid. In general, rather than being “disposed within” the fourth lumen, the non-stretch wire may be embedded within the fourth lumen. In addition, the non-stretch wire may extend to a position adjacent to the distal end region but not fully to the distal end of the inner catheter 14. For example, a short distal segment of the fourth lumen may be filled in with polymer material adjacent to the distal end of the inner catheter 14.

The inner catheter 14 may also include a guidewire tube extension that extends distally from the distal end region. In some embodiments, a nose cone may be attached to the guidewire tube extension. In some embodiments, the nose cone generally is designed to have an atraumatic shape. In some embodiments, the nose cone may also include a ridge or ledge that is configured to abut the distal tip of the outer sheath 12 during delivery of the medical implant 16.

FIG. 2 illustrates some selected components of the medical device system 10 and/or the medical implant 16. For example, here it can be seen that the medical implant 16 may include a plurality of valve leaflets 68 (e.g., bovine pericardial) which may be secured to a cylindrical anchor member or braid 70 that is reversibly actuatable between a “delivery” configuration and a “deployed” configuration. In some embodiments, the medical implant 16 may include at least one locking element 58 configured to lock the anchor member or braid 70 in the “deployed” configuration. In some embodiments, the at least one actuator element (i.e., the push-pull rods 84) may be configured to engage at least one locking element 58 and actuate the anchor member or braid 70 between the “delivery” configuration and the “deployed” configuration. In some embodiments, one actuator element may correspond to, engage with, and/or actuate one locking element 58. In some embodiments, one actuator element may correspond to, engage with, and/or actuate more than one locking element 58. Other configurations are also contemplated.

In some embodiments, the at least one locking element 58 may each comprise a first locking portion such as a post 60, for example at the commissure portions of the valve leaflets 68 (post 60 may sometimes be referred to as a “commissure post”), and a second locking portion such as a buckle 76. In other words, in at least some embodiments, a medical implant 16 may include at least one or a plurality of posts 60 and a corresponding at least one or a plurality of buckles 76. Other configurations and correspondences are also contemplated. In at least some embodiments, the first locking portion may engage the second locking portion in the “deployed” configuration. In some embodiments, the at least one actuator element (i.e., the push-pull rod(s) 84) may include an unlocking member 50 configured to compress the first locking portion of the at least one locking element 58, thereby unlocking the anchor member or braid 70 from the “deployed” configuration.

In some embodiments, the first locking portion may be secured and/or fixedly attached to the anchor member or braid 70. Other embodiments are contemplated where the first locking portion may be movably or removably attached to the anchor member or braid 70. In some embodiments, the second locking portion may be secured and/or fixedly attached to the anchor member or braid 70. Other embodiments are contemplated where the second locking portion may be movably or removably attached to the anchor member or braid 70. In some embodiments, the first locking portion may be fixedly attached to the anchor member or braid 70 and the second locking portion may be fixedly attached to the anchor member or braid 70. In some embodiments, one of the first locking portion and the second locking portion may be fixedly attached to the anchor member or braid 70 and the other may be movably or removably attached to the anchor member or braid 70. In some embodiments, the first locking portion may be movably or removably attached to the anchor member or braid 70 and the second locking portion may be movably or removably attached to the anchor member or braid 70. In some embodiments, the first locking portion may be attached (i.e., fixedly attached, movably attached, removably attached, etc.) to a distal portion of the anchor member or braid 70. In some embodiments, the second locking portion may be attached (i.e., fixedly attached, movably attached, removably attached, etc.) to a proximal portion of the anchor member or braid 70.

In some embodiments, the medical implant 16 may include three individual valve leaflets 68 secured to the anchor member or braid 70 at, adjacent to, and/or using (at least in part) three individual, corresponding posts 60. The valve leaflets 68 may also be secured to a base or “distal end” of the anchor member or braid 70. The posts 60, in turn, may be secured and/or fixedly attached to the anchor member or braid 70 (e.g., along the interior of the anchor member or braid 70) with sutures, adhesives, or other suitable mechanisms. Positioned adjacent to (e.g., aligned with) the plurality of posts 60 are a corresponding plurality of buckles 76, which may also be secured and/or fixedly attached to the anchor member or braid 70 (e.g., along the interior of the anchor member or braid 70) with sutures, adhesives, or other suitable mechanisms. In this example, one buckle 76 is attached to the anchor member or braid 70 adjacent to each of the three posts 60. Accordingly, the anchor member or braid 70 has a total of three buckles 76 and three posts 60 attached thereto. Similarly, one actuating element or push-pull rod 84 may be associated with each post 60 and buckle 76, for a total of three actuating elements or push-pull rods 84 in the illustrated example. Other embodiments are contemplated where fewer or more buckles 76, posts 60, and actuator elements or push-pull rods 84 may be utilized. In some embodiments, a seal 74 (shown in partial cross-section) may be disposed about the anchor member or braid 70 and, as the name suggests, may help to seal the medical implant 16 within a target implant site or area of interest upon deployment.

In some embodiments, attachment between the medical implant 16 and the inner catheter 14 (and/or the outer sheath 12) may be effected through the use of a coupler 78. The coupler 78 may generally include a cylindrical base (not shown) that may be disposed about and/or attached to the inner catheter 14. Projecting distally from the base is a plurality of fingers 79 (e.g., two, three, four, etc.) that are each configured to engage with the medical implant 16 at one of the buckles 76. In some embodiments, each finger 79 may include two self-biased elongated tines 80 (as described further below) configured to engage one buckle 76. A guide 82 may be disposed over each of the fingers 79 and may serve to keep the fingers 79 of the coupler 78 associated with the push-pull rods 84 extending adjacent to (and axially slidable relative to) the fingers 79 of the coupler 78. Finally, a pin release assembly 86 may be a linking structure that keeps posts 60, buckles 76, and push-pull rods 84 associated with one another. In some embodiments, the pin release assembly 86 may include a plurality of individual pins 88 that may be joined together via a coiled connection 90 and held to a pin release mandrel 92 with a ferrule 94.

During delivery, the medical implant 16 may be secured at the distal end of the inner catheter 14 by virtue of the tines 80 of the fingers 79 of the coupler 78 being coupled with corresponding reliefs 77 disposed in a proximal end of the second locking portion or buckle 76, and by virtue of the pins 88 securing together the actuator elements or the push-pull rods 84 and the corresponding first locking portion or posts 60, as will be explained further below. When the medical implant 16 is advanced within the anatomy to the desired location, the outer sheath 12 may be withdrawn (e.g., moved proximally relative to the inner catheter 14) to expose the medical implant 16. Then, the actuator elements or push-pull rods 84 can be used to axially shorten and/or radially expand and “lock” the medical implant 16 and/or the anchor member or braid 70 from the “delivery” configuration (as shown in FIG. 1, for example) to an expanded or “deployed” configuration (as shown in FIG. 2, for example) by proximally retracting the actuator elements or push-pull rods 84 to pull the first locking portions or posts 60 into engagement with the second locking portions or buckles 76. Finally, the pins 88 can be removed, thereby uncoupling the actuator elements or push-pull rods 84 from the first locking portion or posts 60, which allows the push-pull rods 84 and the fingers 79 of the coupler 78 to be withdrawn from the medical implant 16 thereby deploying the medical implant 16 (and/or the anchor member or braid 70) in the anatomy in a “released” configuration (as shown in FIG. 14, for example). In other words, one difference between the “deployed” configuration and the “released” configuration is whether or not the pins 88 are attached to the first locking portions or posts 60. In the “deployed” configuration, the pins 88 are still attached to the first locking portions or posts 60, which thus permits the medical implant 16 (and/or the anchor member or braid 70) to be unlocked via distal advancement of the actuator elements or push-pull rods 84 and the unlocking member 50, as described further below, in order to reposition the medical implant 16, for example.

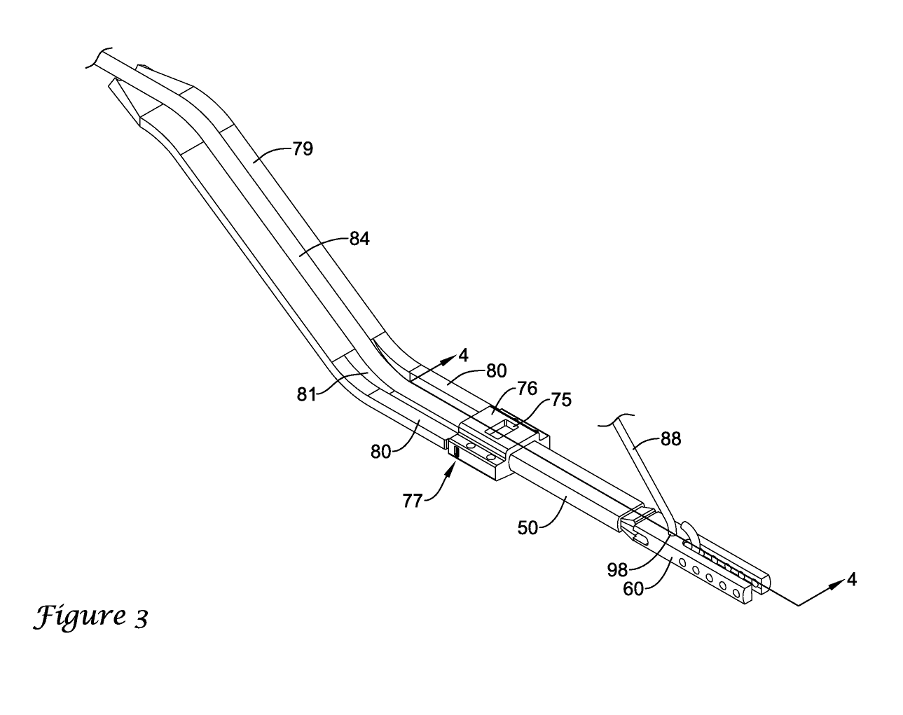

FIGS. 3-10 illustrate selected components of a locking element 58 configured to lock the medical implant 16 (and/or the anchor member or braid 70) in the “deployed” configuration, and the general operation of those components. For simplicity and clarity purposes, only one of the fingers 79 of the coupler 78, only one of the actuator elements or push-pull rods 84, only one of the first locking portions or posts 60, only one of the second locking portions or buckles 76, and only one of the pins 88 are shown and discussed (the whole medical implant 16 and/or the anchor member or braid 70 is not shown to facilitate understanding of the locking element(s) 58). However, it will be understood that the following discussion may apply equally to any and/or all of the components for which there are more than one within the medical implant 16 (i.e., the push-pull rods 84, the buckles 76, the posts 60, the pins 88, etc.) and/or the medical device system 10.

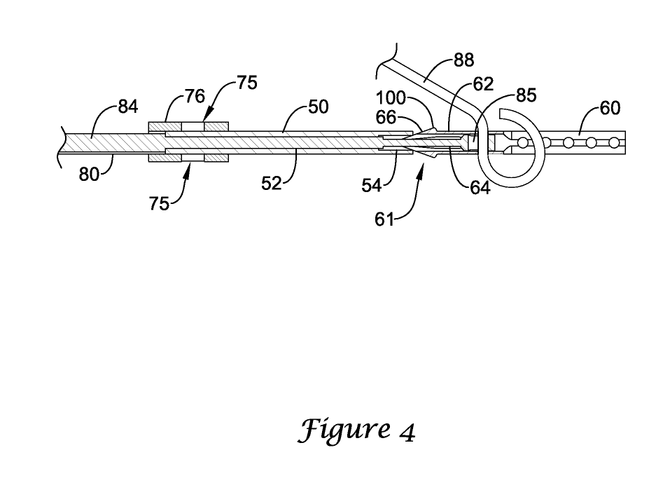

As seen in FIGS. 3-10, each actuator element or push-pull rod 84 extends through a guide 82 adjacent to the finger(s) 79 of the coupler 78, through a second locking portion or buckle 76, and into a passage 64 extending longitudinally through a first locking portion or post 60. The actuator element or push-pull rod 84 may be axially translatable through the second locking portion or buckle 76. In at least some embodiments, the unlocking member 50 may be axially translatable through the second locking portion or buckle 76. A distal end of the actuator element or push-pull rod 84 may include a longitudinally-oriented elongated aperture or slot 85 that can be aligned with an opening 98 through the first locking portion or post 60. When so aligned, a pin 88 can be looped through the opening 98 and the elongated aperture or slot 85 of the actuator element or push-pull rod 84. This releasably couples the actuator element or push-pull rod 84 to the first locking portion or post 60 and forms a configuration of these structures that can be utilized during delivery of the medical implant 16. As can be appreciated, a proximal end of the first locking portion or post 60 and a distal end of the second locking portion or buckle 76 are longitudinally separated (as seen in FIGS. 3 and 4, for example) and, accordingly, the medical implant 16 is in an elongated and generally low-profile “delivery” configuration suitable for percutaneous translation through a patient's anatomy to an area of interest and/or target site.

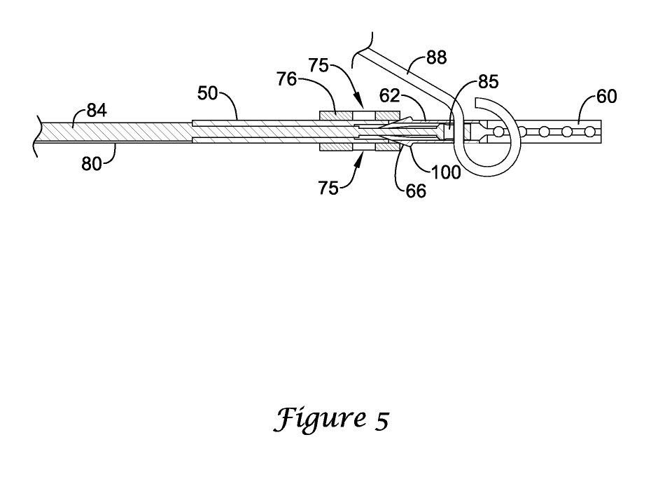

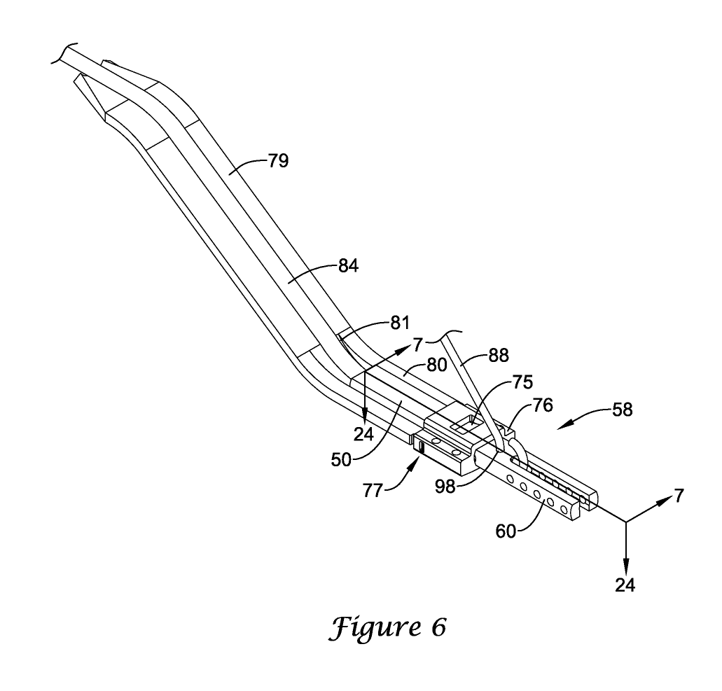

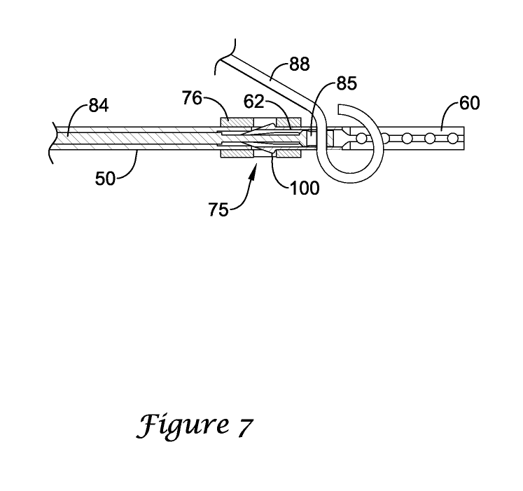

When medical implant 16 reaches the intended target site within the anatomy, a clinician can proximally retract the actuator element or push-pull rod 84, thereby moving a proximal end of the first locking portion or post 60 toward a distal end of the second locking portion or buckle 76 (as seen in FIG. 5, for example) in order to axially shorten and/or radially expand the anchor member or braid 70 of the medical implant 16 towards the “deployed” configuration. When retracting or pulling the actuator element or push-pull rod 84 proximally, the pin 88, positioned through the opening 98 in the first locking portion or post 60, is disposed through a distal end of the elongated aperture or slot 85. Ultimately, the actuator element or push-pull rod 84 can be retracted sufficiently far enough to lock the first locking portion or post 60 with the second locking portion or buckle 76 so as to lock the medical implant 16 in the “deployed” configuration (as seen in FIGS. 6 and 7, for example), suitable for implantation within the anatomy. In other words, in some embodiments, axial translation of the actuator element or push-pull rod 84 in a first (e.g., proximal) direction may actuate the anchor member or braid 70 from the “delivery” configuration to the “deployed” configuration.

FIG. 5 illustrates the actuator element or push-pull rod 84 proximally retracted such that a second end of the tapered end portion 61 of a plurality of legs 62 of the first locking portion or post 60 (described in more detail below) is pulled into a passageway 72 extending longitudinally through the second locking portion or buckle 76 (also described in more detail below) until one or more angled surfaces 66 on the tapered end portion 61 of the plurality of legs 62 of the first locking portion or post 60 is brought into contact with a distal end of the second locking portion or buckle 76. At this point, the plurality of legs 62 of the first locking portion or post 60 may deflect inward toward each other as the one or more angled surfaces 66 contact the distal end of the second locking portion or buckle 76 while being pulled proximally into the passageway 72 extending longitudinally through the second locking portion or buckle 76.

In doing so, a raised, generally transversely-oriented shoulder or ridge 100 disposed on a first end of the tapered end portion 61 of the plurality of the legs 62 of the first locking portion or post 60 may be pulled proximally into the passageway 72 extending longitudinally through the second locking portion or buckle 76 until the raised, generally transversely-oriented shoulder or ridge 100 reaches at least one recess 75 extending outward from the passageway 72. While passing through the passageway 72, the plurality of legs 62 remain deflected inward by a compressive force applied to the shoulder or ridge 100 by an interior surface of the second locking portion or buckle 76. Upon reaching the at least one recess 75, the compressive force is removed from the shoulder or ridge 100, and the plurality of legs 62 rebound and/or extend outwardly toward an unstressed position as the shoulder or ridge 100 engages and/or extends into the at least one recess 75 (as seen in FIGS. 6 and 7, for example). With the shoulder or ridge 100 engaged with and/or disposed within the at least one recess 75, the first locking portion or post 60 is secured to and/or locked to the second locking portion or buckle 76 such that the anchor member or braid 70 is locked in the “deployed” configuration.

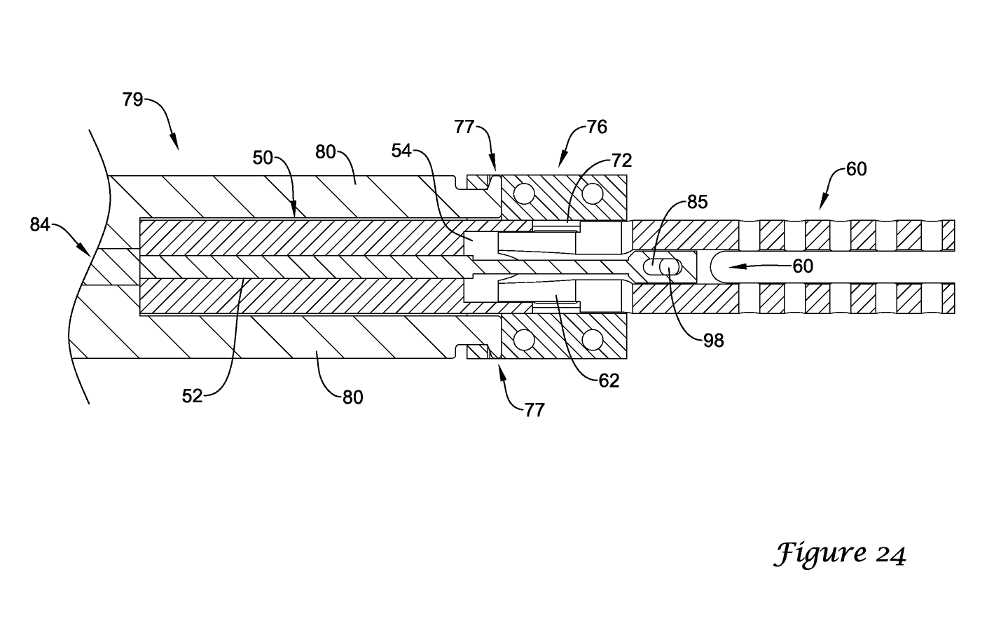

It should be noted that in the “delivery” configuration and in the “deployed” configuration, the unlocking member 50 is positioned between the tines 80 of the finger 79 of the coupler 78. As will be described below, the tines 80 of each finger 79 are self-biased inwardly toward a centerline of the finger 79, and will have a tendency to bend inward unless forced or held outward from the centerline of the finger 79. The unlocking member 50 is positioned between the tines 80 to prevent the tines 80 from bending inwardly toward the centerline of the finger 79. While the unlocking member 50 is positioned between the tines 80, a notch or hook member at a distal end of each tine 80 is engaged with and/or disposed within a relief 77 within a proximal end of the second locking portion or buckle 76 (as seen in FIG. 24, for example), thereby attaching and/or securing the medical implant 16 to the inner catheter 14 and permitting axial translation of the actuator element or push-pull rod 84 relative to the second locking portion or buckle 76.

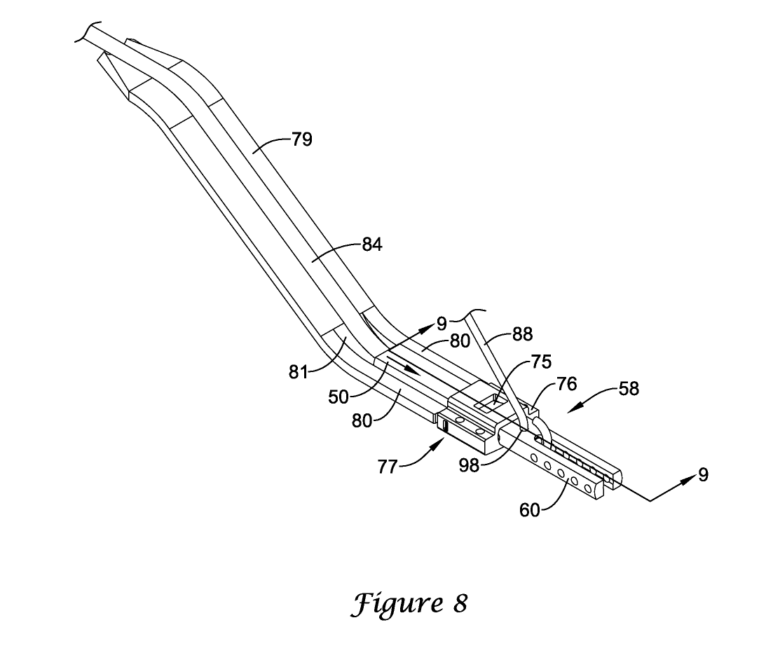

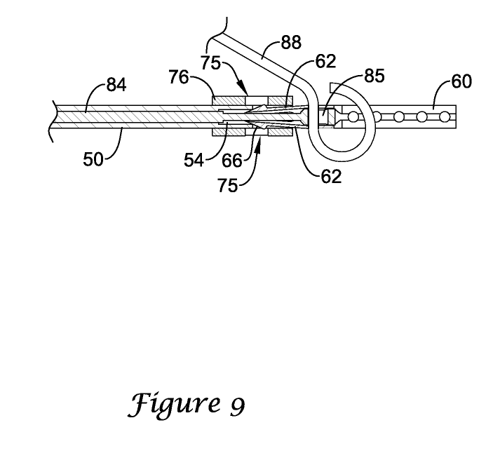

In some embodiments and/or some procedures, it may be desirable to unlock the anchor member or braid 70 from the “deployed” configuration and extend the anchor member or braid 70 back toward the “delivery” configuration in order to reposition or retract/remove the medical implant 16, for example. To do so, a clinician may urge the actuator element or push-pull rod 84 distally to “unlock” the anchor member or braid 70 or the medical implant 16, as seen in FIG. 8, for example. When the actuator element or push-pull rod 84 is urged distally, the actuator element or push-pull rod 84 may translate distally relative to the at least one locking element 58 (i.e., the first locking portion or post 60 and/or the second locking portion or buckle 76), so as to position the pin 88 extending through the opening 98 in a proximal end of the elongated aperture or slot 85, and to receive the tapered end portion 61 deeper into the cavity 54 of the unlocking member 50 which will compress the first locking portion or post 60 of the at least one locking element 58 toward the actuator element or push-pull rod 84, as seen in FIG. 9, for example. In other words, axial translation of the actuator element or push-pull rod 84 in a second direction opposite from the first direction engages the unlocking member 50 with the tapered end portion 61 to unlock the anchor member or braid 70 from the “deployed” configuration.

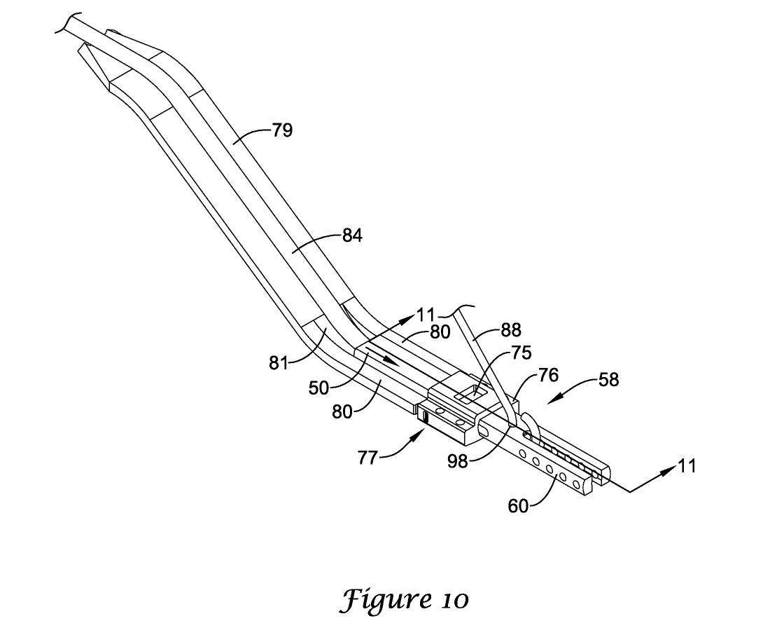

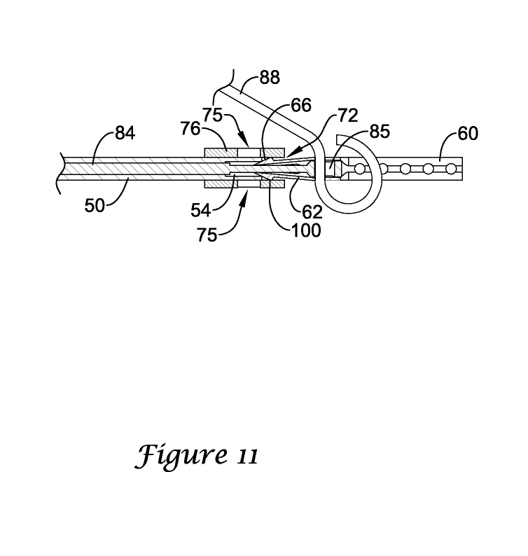

With the pin 88 positioned at the proximal end of the elongate aperture or slot 85, further distal urging of the actuator element or push-pull rod 84 will no longer cause translation of the actuator element or push-pull rod 84 relative to the at least one locking element 58 as a whole. Instead, further distal urging (i.e., axial translation in the second direction) of the actuator element or push-pull rod 84 will result in the actuator element or push-pull rod 84 and the first locking portion or post 60, joined together by the pin 88, to translate distally relative to the second locking portion or buckle 76, thereby actuating the anchor member or braid 70 from the “deployed” configuration toward the “delivery” configuration, as seen in FIGS. 10 and 11, for example. In other words, distal axial translation of the actuator element or push-pull rod 84 may compress the first locking portion or post 60 of the at least one locking element 58 laterally inward, thereby disengaging the first locking portion or post 60 from the second locking portion or buckle 76 of the at least one locking element 58 fixedly attached to the anchor member or braid 70.

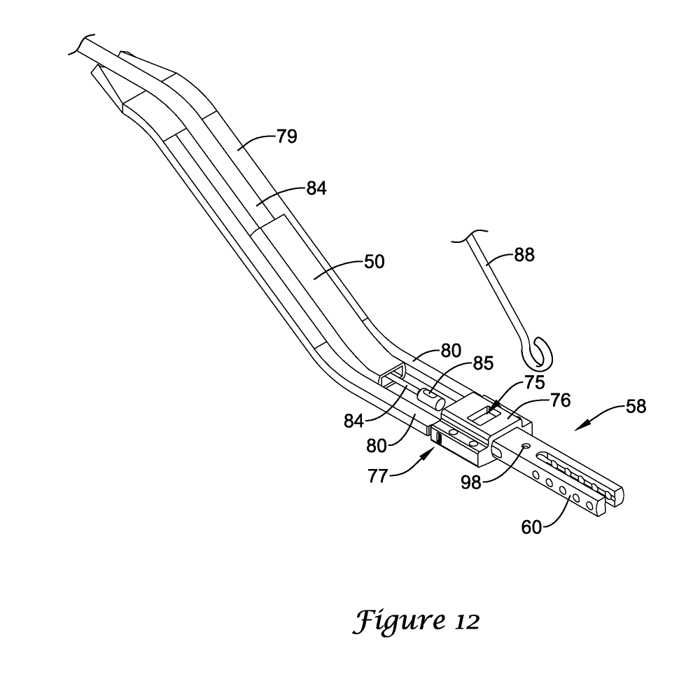

Alternatively, if a clinician is satisfied with the positioning and/or locking of the medical implant 16 (e.g., after visualization of the medical implant 16 via a suitable imaging technique), the pin 88 may be pulled (e.g., removed from opening 98 and the elongated aperture or slot 85 at the distal end of the actuator element or push-pull rod 84) to uncouple and/or disengage the actuator element or push-pull rod 84 from the first locking portion or post 60, thereby permitting proximal retraction of the actuator element or push-pull rod 84 from the at least one locking element 58, as seen in FIG. 12, for example. Retraction of the actuator element or push-pull rod 84 from the at least one locking element 58 causes engagement of the unlocking member 50 with a tapered ramp 81 formed in the finger 79 between the tines 80 which deflects the unlocking member 50 along the finger 79 and within the guide 82 (not shown in FIG. 12). As can be seen in FIG. 12, the unlocking member 50 and/or the actuator element or push-pull rod 84 are sufficiently flexible to bend and/or track along a surface of the finger 79 and within the guide 82 without adding unnecessary friction and/or resistance. In at least some embodiments, the finger 79 and/or the unlocking member 50 may include a lubricious coating disposed thereon to further facilitate translational movement therebetween.

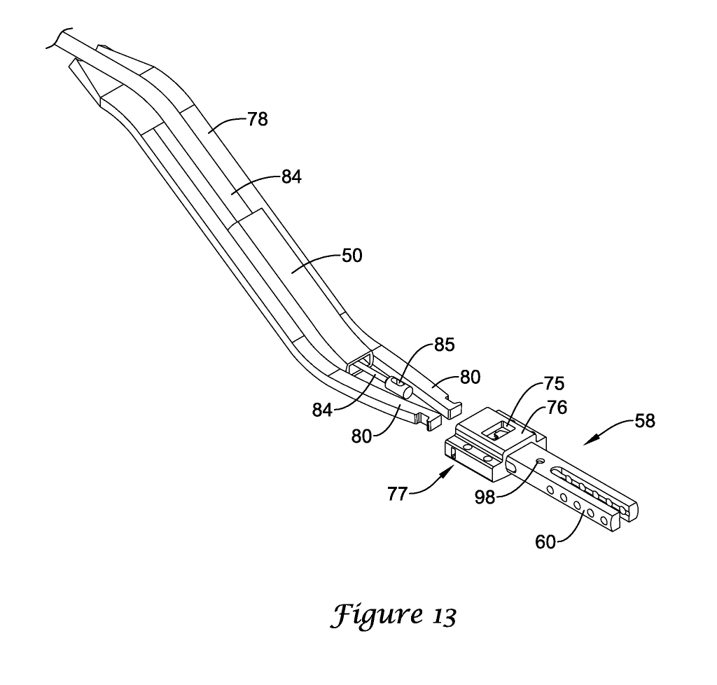

As the unlocking member 50 is retracted from between the tines 80 by the actuator element or push-pull rod 84, the self-biased tines 80 may begin to bend inward toward each other, thereby retracting the notch or hook member from the relief(s) 77. After the notch or hook member on each tine 80 has disengage the relief(s) 77, further proximal retraction of the at least one actuator element or push-pull rod 84 causes the finger(s) 79 of the coupler 78 to retract proximally from the at least one locking element 58 and the medical implant 16, as seen in FIG. 13, for example, thereby leaving the medical implant 16 disposed at the target site in the “released” configuration, shown in FIG. 14, for example.

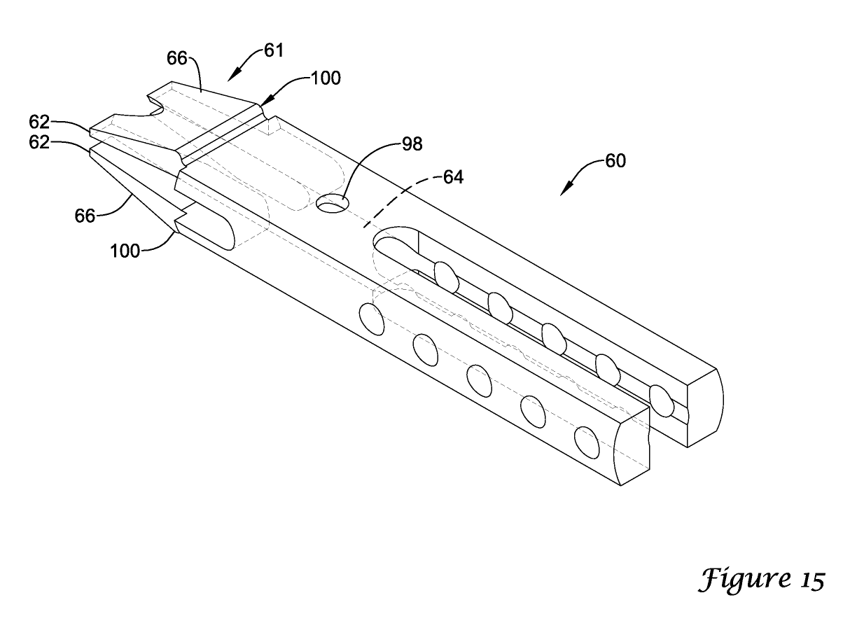

FIG. 15 illustrates an example of the first locking portion or post 60 of the at least one locking element 58. As seen in the figure, the first locking portion or post 60 may include an elongated member having a longitudinally-extending passage 64 extending therethrough and an opening 98 extending transversely therethrough for receiving a pin 88. In some embodiments, the elongated member of the first locking portion or post 60 may include at least two openings 98 positioned on opposing sides of the elongated member and/or the passage 64. In some embodiments, the actuator element or push-pull rod 84 may be slidably disposed within with passage 64 and be releasably coupled to the first locking portion or post 60 by the pin 88 disposed through the opening(s) 98 and the elongated aperture or slot 85 in the distal end of the actuator element or push-pull rod 84. In some embodiments, the first locking portion or post 60 may include one or more apertures or other features provided to aid in attaching the first locking portion or post 60 to the anchor member or braid 70.

In at least some embodiments, the first locking portion or post 60 may include a tapered end portion 61 having a first end and a second end. In some embodiments, the tapered end portion 61 may be configured to engage a circumferential frame of the second locking portion or buckle 76, described in more detail below. In some embodiments, the tapered end portion 61 may be tapered from a first outer extent at the first end to a second outer extent at the second end, wherein the second outer extent is less than the first outer extent when the tapered end portion 61 is in an unstressed or relaxed configuration. In some embodiments, the first end of the tapered end portion 61 may define a shoulder or ridge 100 oriented generally transverse to a longitudinal axis of the elongated member of the first locking portion or post 60. In at least some embodiments, the shoulder or ridge 100 may extend outward from a body of the first locking portion or post 60 and/or may have a greater outer extent than the body of the first locking portion or post 60.

In some embodiments, the tapered end portion 61 may be configured to flex or deflect inwardly as the tapered end portion 61 is translated through a passageway 72 of the second locking portion or buckle 76. In some embodiments, the tapered end portion 61 may include a plurality of legs 62 configured to flex or deflect inwardly toward each other as the tapered end portion 61 is translated through a passageway of the second locking portion or buckle 76. In some embodiments, the tapered end portion 61 may include a tapered surface 66 on each of the plurality of legs 62, the tapered surface 66 extending from the first end to the second end of the tapered end portion 61. In at least some embodiments, the tapered end portion 61 may be disposed at a proximal end of the first locking portion or post 60, as the first locking portion or post 60 is positioned and/or attached to the anchor member or braid 70. In at least some embodiments, the tapered surface(s) 66 may form a wedge-like shape having a narrow end (as viewed transversely or from the side) at the proximal end of the first locking portion or post 60 and a wide end distal of the narrow end. As will be apparent, the tapered surface(s) 66 of the tapered end portion 61 may engage a distal end of the second locking portion or buckle 76 and urge and/or deflect the tapered end portion 61 inwardly as the first locking portion or post 60 is translated proximally relative to the second locking portion or buckle 76 as a result of proximal retraction of the actuator element or push-pull rod 84.

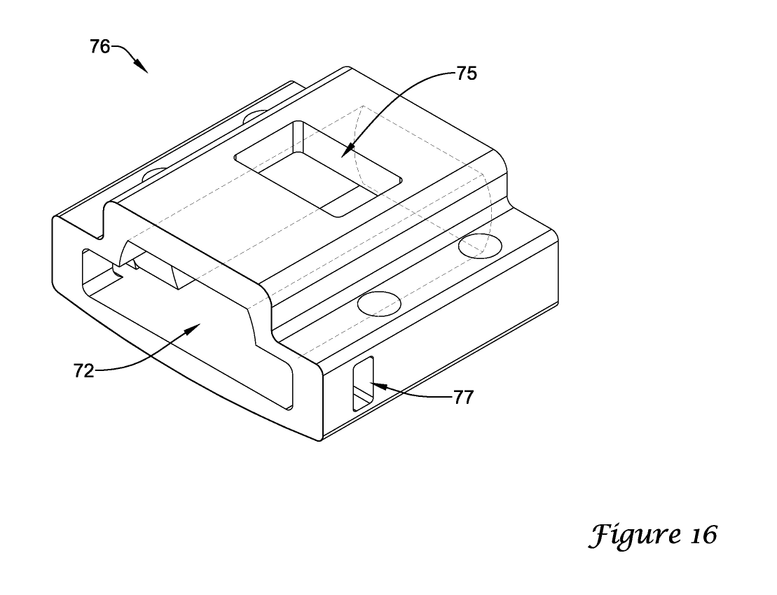

FIG. 16 illustrates an example of the second locking portion or buckle 76 of the at least one locking element 58. In at least some embodiments, the second locking portion or buckle 76 may include a circumferential frame defining a passageway 72 extending longitudinally therethrough. In some embodiments, the passageway 72 may be configured to receive at least a portion of the elongated member of the first locking portion or post 60 therein. In some embodiments, the circumferential frame may be configured to engage the tapered end portion 61 when the elongated member of the first locking portion or post 60 is translated through the passageway 72. In some embodiments, the second locking portion or buckle 76 may include one or more apertures or other features provided to aid in attaching the second locking portion or buckle 76 to the anchor member or braid 70.

In some embodiments, the circumferential frame of the second locking portion or buckle 76 may include at least one recess 75 extending outwardly from the passageway 72 configured to receive and/or engage the shoulder or ridge 100 of the tapered end portion 61 of the first locking portion or post 60. In some embodiments, the circumferential frame of the second locking portion or buckle 76 may include two recesses 75 disposed on opposing sides of the second locking portion or buckle 76. Additional recesses 75 and/or other arrangements are also contemplated. In some embodiments, the at least one recess 75 may extend outward from the passageway 72 completely through a wall of the second locking portion or buckle 76. In some embodiments, the at least one recess 75 may extend outward from the passageway 72 partially through a wall of the second locking portion or buckle 76. Various combinations of these arrangements are also contemplated. In some embodiments, translating the second end of the tapered end portion 61 through the passageway 72 of the second locking portion or buckle 76 until the shoulder or ridge 100 has engaged the at least one recess 75 locks the anchor member or braid 70 in the “deployed” configuration. In some embodiments, the actuator element or push-pull rod 84 extends through the passageway 72 of the second locking portion or buckle 76 and is releasably coupled to the first locking portion or post 60. In at least some embodiments, the actuator element or push-pull rod 84 may be axially translatable through the second locking portion or buckle 76.

In some embodiments, the circumferential frame of the second locking portion or buckle 76 may include at least two reliefs 77 extending outwardly from the passageway 72. In some embodiments, the at least two reliefs 77 may be disposed on opposite sides of the second locking portion or buckle 76. In some embodiments, a notch or hook member at a distal end of the tines 80 of each finger 79 of the coupler 78, described in more detail below, may be configured to engage with a relief 77, thereby attaching the anchor member or braid 70 (or the medical implant 16) to the inner catheter 14, when the unlocking member 50 is disposed between the tines 80 such as when the actuator element or push-pull rod 84 is releasably coupled to the first locking portion or post 60. In some embodiments, the at least two reliefs 77 may extend outward from the passageway 72 completely through a wall of the second locking portion or buckle 76. In some embodiments, the at least two reliefs 77 may extend outward from the passageway 72 partially through a wall of the second locking portion or buckle 76. Various combinations of these arrangements are also contemplated.

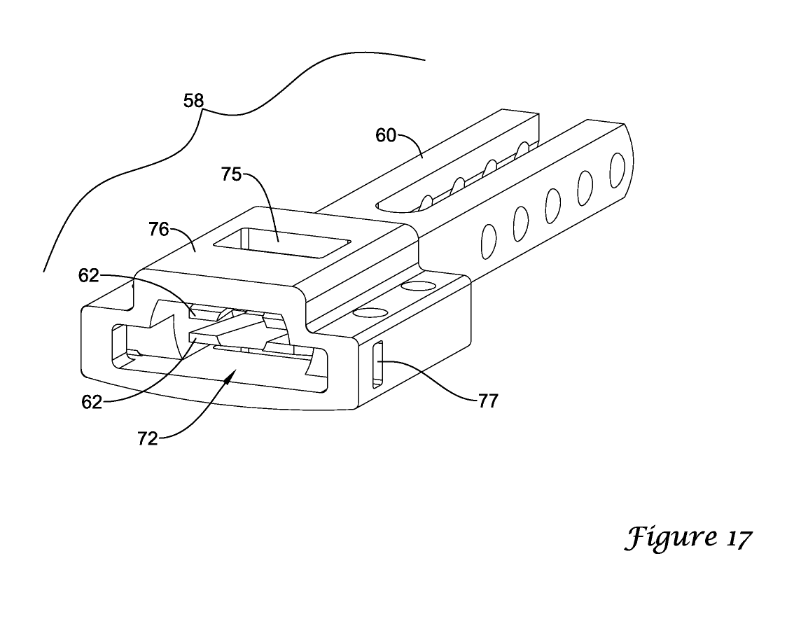

FIG. 17 illustrates a partial exemplary view of the at least one locking element 58, including the first locking portion or post 60 engaged with and/or locked to the second locking portion or buckle 76. As can be seen in the figure, the legs 62 of the tapered end portion 61 may be disposed within the passageway 72. When the shoulder or ridge 100 engages and/or extends into the at least one recess 75, the anchor member or braid 70 will have assumed the “deployed” configuration, as discussed above.

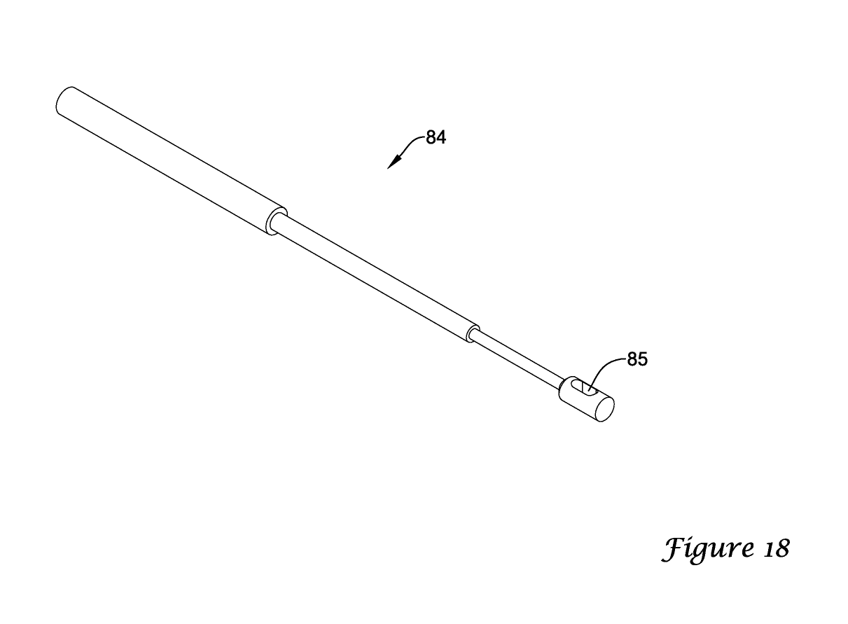

FIG. 18 illustrates an example actuator element or push-pull rod 84. In at least some embodiments, the actuator element or push-pull rod 84 may be formed from a solid wire or rod. In some embodiments, at least a portion of the solid wire or rod may be machined, ground, or otherwise had material removed or added to form a stepped structure, as shown. In some embodiments, the actuator element or push-pull rod 84 may be tapered instead of stepped. Various combinations of these configurations are also contemplated. In some embodiments, the actuator element or push-pull rod 84 may include an elongated aperture or slot 85 extending transversely through the actuator element or push-pull rod 84 at a distal end thereof. In at least some embodiments, the actuator element or push-pull rod 84 may be releasably engaged to the first locking portion or post 60 by a pin 88 disposed within the elongated aperture or slot 85.

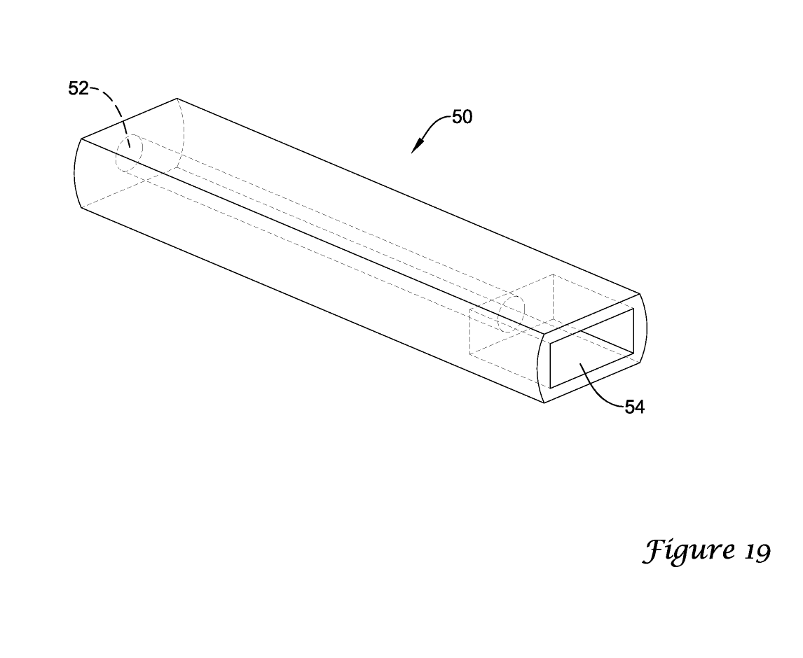

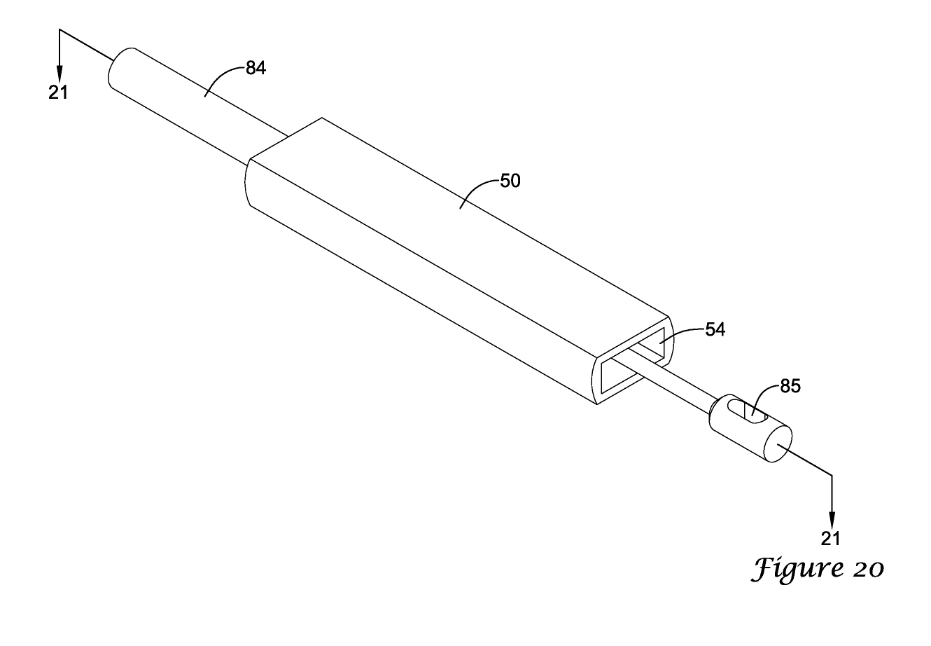

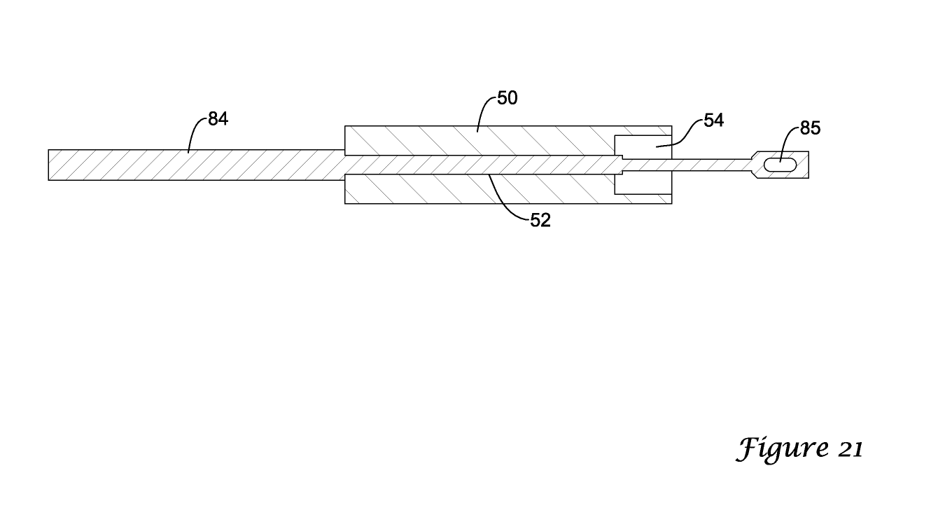

FIG. 19 illustrates an example unlocking member 50. In some embodiments, the unlocking member 50 may include a lumen 52 extending longitudinally therethrough and a distally-facing cavity 54 formed within a distal end of the unlocking member 50. In some embodiments, the lumen 52 may extend from the cavity 54 through a proximal end of the unlocking member 50. In at least some embodiments, the cavity 54 may be configured to receive at least a portion of the first locking portion or post 60 therein. In some embodiments, the cavity 54 may be configured to receive at least a portion of the tapered end portion 61 and/or the legs 62. In some embodiments, the tapered surface 66 of the tapered end portion 61 of the first locking portion or post 60 may engage a distal end of the unlocking member 50 as the second end of the tapered end portion is received within the cavity 54. In some embodiments, the actuator element or push-pull rod 84 may be disposed within the lumen 52. In at least some of those embodiments, the actuator element or push-pull rod 84 may be fixedly attached to the unlocking member 50, such as by welding, brazing, adhesives, friction, interference fit, or other methods or means known in the art, as seen in FIGS. 20 and 21, for example. In some embodiments, the unlocking member 50 is axially translatable through the passageway 72 (i.e., within the circumferential frame) of the second locking portion or buckle 76. In some embodiments, an outer profile of the unlocking member 50 and a shape or profile of the passageway 72 of the second locking portion or buckle 76 may be complimentary and/or prevent relative rotational movement thereof. In other words, the unlocking member 50 may be prevented from rotating relative to the second locking portion or buckle 76, thereby maintaining a preferred relative orientation between them unlocking member 50 and the second lucking portion or buckle 76.

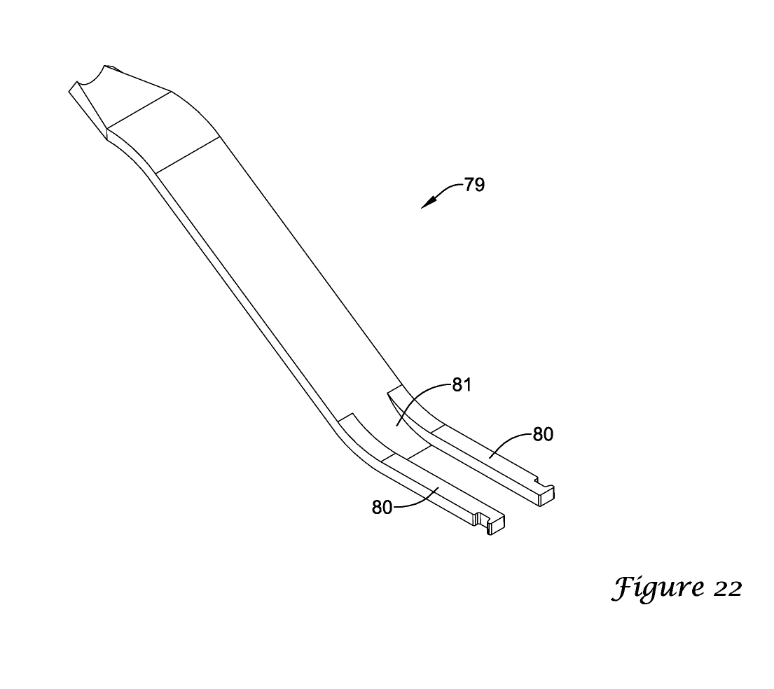

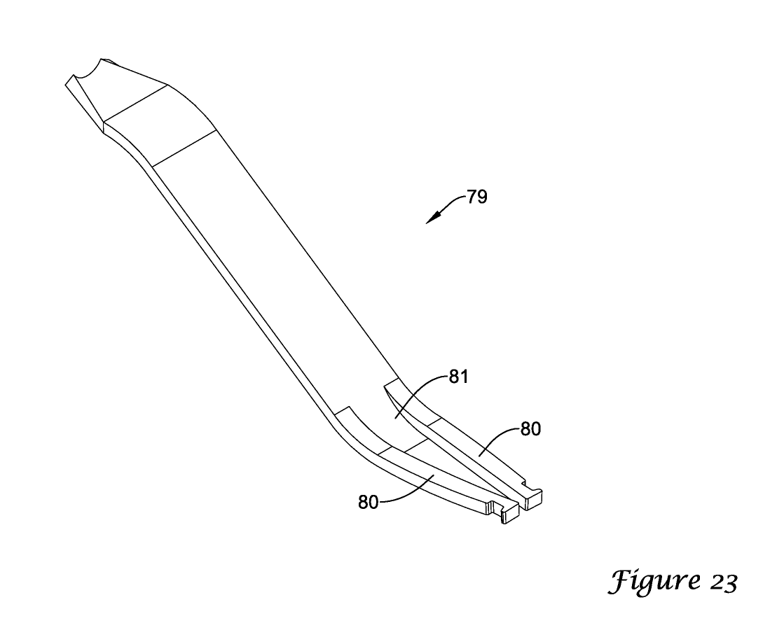

FIGS. 22 and 23 illustrate an example finger 79 of the coupler 78. In some embodiments, the finger 79 may include two elongated tines 80 extending distally at a distal end thereof. In some embodiments, a distal end of each of the elongated tines 80 may include a notch or hook member configured to couple to and/or engage with the reliefs 77 of the second locking portion or buckle 76. The two elongated tines 80 may be spaced apart from each other sufficiently to permit the unlocking member 50 to be disposed therebetween, as seen in FIG. 24, for example. In some embodiments, the finger 79 may include a ramp 81 disposed adjacent a distal end thereof, the ramp 81 being configured to guide the unlocking member 50 along the finger 79 and into a guide 82 disposed about the finger 79. In use, the unlocking member 50 may be disposed between the tines 80 of the finger 79, thereby maintaining the tines 80 in a spaced apart relationship, such as that shown in FIG. 22. In a spaced apart position, the tines 80 may couple to and/or engage with the reliefs 77 of the second locking portion or buckle 76, to attach the anchor member or braid 70 and/or the medical implant 16 to the inner catheter 14 and to facilitate movement and/or manipulation the anchor member or braid 70 and/or the medical implant 16.

In at least some embodiments, the tines 80 may be self-biased inwardly toward each other, as seen in FIG. 23. Upon removal of the unlocking member 50 from between the tines 80 by proximal retraction or withdrawal of the actuator element or push-pull rod 84, the tines 80 may bend toward each other to facilitate releasing the anchor member or braid 70 and/or the medical implant 16 at the target site.

In some embodiments, the handle 18 may include a handle housing. A rotatable control knob may be disposed about the handle housing (e.g., at a proximal end of the handle housing) and may be used to move one or more of the components of the medical device system 10 (e.g., outer sheath 12, push-pull rods 84, etc.). A rotatable collar may be disposed about the handle housing. The control knob may be disposed about a proximal portion of the rotatable collar. A slidable door may also be disposed about the handle housing. The slidable door may translate distally to expose a distal portion of the rotatable collar positioned generally under the slidable door. The rotatable collar may be rotated to move one or more components of the medical device system 10 (e.g., push-pull rods 84, pin release mandrel 92, etc.). The handle 18 may also include one or more apertures and/or flush ports that can be used to flush the medical device system 10. In some embodiments, a distal flush port and a proximal flush port may be accessible from the exterior of the handle housing through a distal aperture and a proximal aperture, respectively.

A proximal end of the inner catheter 14 may be attached (e.g., fixedly attached) to an interior body or diverter. The diverter may be attached to a support body. In general, the diverter and/or the support body may have one or more passageways or lumens formed therein. In some embodiments, the actuator elements or push-pull rods 84 and/or the pin release mandrel 92 may extend through respective passageways. Alternatively, the proximal ends of the actuator elements or push-pull rods 84 and/or the pin release mandrel 92 may each be attached to a shaft or hypotube (e.g., solid in cross-section, tubular, etc.), and each of the shafts may extend through the one or more passageways. For example, a first shaft or hypotube and a second shaft or hypotube may extend through the passageways in the diverter, and in some embodiments, the first shaft or hypotube extends through a first passageway and the second shaft or hypotube extends through a second passageway that is separate or distinct from the first passageway. In at least some embodiments, the first shaft is attached to the pin release mandrel 92. In at least some embodiments, the second shaft is attached to the actuator elements or push-pull rods 84. As noted above, in least some embodiments of the medical device system 10, three actuator elements or push-pull rods 84 are utilized. In these embodiments, the three actuator elements or push-pull rods 84 come together (e.g., brought into contact with one another or otherwise brought into relatively close proximity with one another) adjacent to the distal end of the inner catheter 14 and enter the first lumen. At one or more positions along their length, the actuator elements or push-pull rods 84 may be attached to one another. For example, in some embodiments, the actuator elements or push-pull rods 84 may be welded together about 10.16 cm (about 4.00 inches) from their distal ends. In some embodiments, actuator elements or push-pull rods 84 may be welded together proximate their proximal ends in addition to or instead of the distal weld. Proximally thereafter, the actuator elements or push-pull rods 84 may extend to the second shaft.

A hypotube (e.g., a hypotube liner disposed along a guidewire lumen) may extend through the diverter within a passageway therein and then be “diverted” around a portion of the diverter and the support body, and ultimately be extended to a position at the proximal end of the handle 18 so as to provide a user access to the guidewire lumen. The proximal flush port may be disposed on the support body that can be used to flush the lumens of the inner catheter 14 and, for example, may function similarly to the distal flush port.

At their respective proximal ends, the first shaft may be secured to a slider and the second shaft may be secured to a force limiter body. The connections between the various components may include a number of different types of connections including mechanical bonding (e.g., pinning, threading, interference fit, etc.), adhesive bonding, thermal bonding, etc. The slider may be slidable relative to the force limiter body. In some embodiments, the slider may be selectively locked to the force limiter body, thereby preventing relative movement between the slider and the force limiter body. The force limiter body may be secured to a push-pull rod carriage, which may be threaded onto a lead screw. Thus, movement of the lead screw can cause movement of the push-pull rod carriage and the force limiter body and thus, the actuator elements or push-pull rods 84 (via the second shaft).

In general, the force limiter body forms or defines a stop point that provides tactile feedback (e.g., resistance to further rotation of the control knob) to the user indicating that the actuator elements or push-pull rods 84 have been retracted proximally a sufficient distance to lock the posts 60 with the buckles 76. To verify proper locking, a clinician may use an appropriate visualization technique to visualize proper locking (e.g., the relative positioning of the posts 60 and the buckles 76). A chock may be positioned adjacent to the slider to selectively lock the slider to the force limiter body. In order to allow the pin release mandrel 92 to be proximally retracted to pull the pins 88, the chock can be rotated or otherwise moved to a secondary position or configuration. When in this configuration, the chock no longer forms a barrier to further movement of, for example, the slider and the pin release mandrel 92. Accordingly, with the chock no longer acting as an impediment, the slider and the pin release mandrel 92 can be proximally retracted to facilitate deployment of the medical implant 16 by allowing the pins 88 to be pulled.

The handle 18 also includes a rotatable ring with internal teeth that are configured to engage with teeth on a gear coupled to the lead screw. The ring is coupled to the control knob so that rotation of the control knob results in analogous motion of the ring and thus the lead screw.

The handle 18 is generally configured for coordinated movement of multiple structures of the medical device system 10. For example, the handle 18 is configured to allow a user to move the outer sheath 12 (e.g., relative to the inner catheter 14), move the actuator elements or push-pull rods 84, and move the pin release mandrel 92. Moreover, the handle 18 is configured so that the appropriate structure can be moved at the appropriate time during the intervention so that the medical implant 16 can be delivered and released in an efficient manner. Some examples of how the coordinated movement of the medical device system 10 may occur within the handle 18 may be similar to those disclosed in U.S. Patent Application Publication No. US 2010/0280495, the entire disclosure of which is herein incorporated by reference.

To help facilitate the coordinated movement, the handle 18 may include a lost motion barrel. The lost motion barrel is configured to engage a sheath carriage and a push-pull rod carriage and/or screws associated with the sheath and push-pull rod carriages at different times during the intervention to stop motion (e.g., create “lost motion” of the appropriate carriage). For example, in a first position or state for the handle 18, the outer sheath 12 is extended distally relative to the inner catheter 14 (and the handle 18) so as to fully sheath (e.g., contain) the medical implant 16. While in this position, the sheath carriage is positioned adjacent to the distal end of the handle 18. In addition, a rod screw associated with the push-pull rod carriage is extended distally from the push-pull rod carriage and positioned within a lost motion barrel. Upon rotation of the control knob (e.g., in the clockwise direction), the lead screw begins to rotate. Rotation of the lead screw causes the sheath carriage to move along the lead screw in the proximal direction, resulting in proximal movement of the outer sheath 12 (e.g., “unsheathing” the medical implant 16). This initial rotation of the lead screw also causes a rod screw to rotate. This may be because, for example, a knob or projection (not shown) on the rod screw may be engaged with a helical thread disposed along the interior of the lost motion barrel. However, because the rod screw is spaced from the push-pull rod carriage, it does not exert a force onto the push-pull rod carriage. Thus, initial motion of the control knob does not result in movement of the push-pull rod carriage and, instead, only results in translation of the sheath carriage and rotation (and translation) of the rod screw.

Eventually, the rod screw (e.g., the knob formed therein) reaches an essentially linear thread or pathway formed at the end of the lost motion barrel. The linear thread allows the rod screw to translate along the lead screw to a position where the rod screw contacts (e.g., is threaded within and abuts) the push-pull rod carriage. In doing so, the rod screw can contact and move the proximally push-pull carriage. Accordingly, further rotation of the lead screw not only causes the sheath carriage to move proximally but also causes the push-pull rod carriage to move proximally.

When the sheath carriage reaches the lost motion barrel, a sheath carriage screw of the sheath carriage enters the lost motion barrel. This may occur in a manner similar to how the rod screw threads and unthreads with the helical thread formed along the lost motion barrel. For example, while the sheath carriage is translating, the sheath carriage screw may follow an essentially linear thread or pathway formed along or adjacent to the lost motion barrel. Upon reaching the lost motion barrel, the sheath carriage screw (e.g., a knob or projection formed thereon) may shift into engagement with the helical thread within the lost motion barrel and rotate. This rotation “unthreads” the sheath carriage screw from the sheath carriage. Accordingly, additional rotation of the lead screw results in continued proximal movement of the push-pull rod carriage while motion of the sheath carriage ceases.

In at least some embodiments, the lead screw has a plurality of portions, for example a first portion and a second portion, with a differing pitch to its thread. This may allow the sheath and push-pull rod carriages to travel at different rates along the lead screw. For example, the pitch of the lead screw along which the sheath carriage translates may be generally more spaced or slanted than at positions adjacent to the push-pull rod carriage. Accordingly, the coordinated movement of the sheath and push-pull rod carriages also may be configured so that the sheath carriage translates along the lead screw at a greater rate than the push-pull rod carriage. Other configurations are contemplated where the above-mentioned configuration is reversed as well as further configurations where the pitch of the lead screw is essentially constant or includes a number of different pitch regions.

Sufficient proximal retraction of the push-pull rod carriage may result in the actuator elements or push-pull rods 84 being sufficiently retracted so that the posts 60 can engage and lock with the buckles 76. When the clinician is satisfied that locking is complete (e.g., after verification via an appropriate visualization technique), the clinician may proximally retract the pin release mandrel 92 in order to pull the pins 88 from the openings 98 and the elongated apertures or slots 85 in the actuator elements or push-pull rods 84 to release the medical implant 16.

To initiate release of the pins 88, the door may be slid distally along a collar which is positioned on the handle 18. When the door is sufficiently advanced, the door and the collar, together, can be rotated about a longitudinal axis of the handle 18. The push-pull rod carriage may also include a radially-extending proximal flag member. In general, the flag member may be designed as a feature that can prevent the collar from being rotated earlier than desired (and, thus, prevent the pins from being pulled earlier than desired). For example, the flag member may be positioned within and follow a groove along the interior of the collar. While positioned within the groove, the flag member essentially forms a physical barrier that prevents the collar from rotating relative to the handle housing. When the push-pull rod carriage is translated proximally to the back of the handle housing (e.g., when the actuator elements or push-pull rods 84 are proximally retracted so as to lock the posts 60 with the buckles 76), the flag member exits the groove in the collar. Accordingly, the flag member no longer impedes rotation of the collar and, as such, the collar can now be rotated to pull the pins 88.

The collar, via a ring, is associated with a gear engaged with a secondary screw. Notches at a proximal end of the collar engage protrusions on the ring such that rotation of the collar causes corresponding rotation of the ring and thus the secondary screw. The initial rotation of the collar is sufficient to rotate the chock (e.g., via a mechanical interaction between the collar and the chock that causes the chock to shift) from a first configuration where the slider (and, thus, the pin release mandrel 92) is selectively locked to the force limiter body, to a secondary configuration, which permits the slider to translate along the secondary screw as the secondary screw rotates, to proximally retract and pull the pins 88 (e.g., via the pin release mandrel 92). The chock in the first configuration engages a ridge along a top portion of the force limiter body which forms a physical barrier that prevents proximal translation of the slider relative to the force limiter body. When the collar is rotated to shift the chock into the secondary configuration, the slider can translate proximally within a groove disposed in the top portion of the force limiter body, as the collar is rotated about the handle housing to pull the pins 88 from the openings 98 and the elongate apertures or slots 85 in the distal ends of the actuator elements or push-pull rods 84. Once the pins 88 have been removed, the actuator elements or push-pull rods 84 may be withdrawn from the medical implant 16, thereby releasing the implant at the target site (area of interest).

Following release of the medical implant 16, the control knob may be rotated to move the sheath carriage distally within the handle housing, thereby moving the outer sheath 12 distally relative to the inner catheter 14 and the coupler 78 so as to cover or re-sheath the elements of the medical device system 10 disposed at the distal end. The medical device system 10 may then be removed from the patient's anatomy.

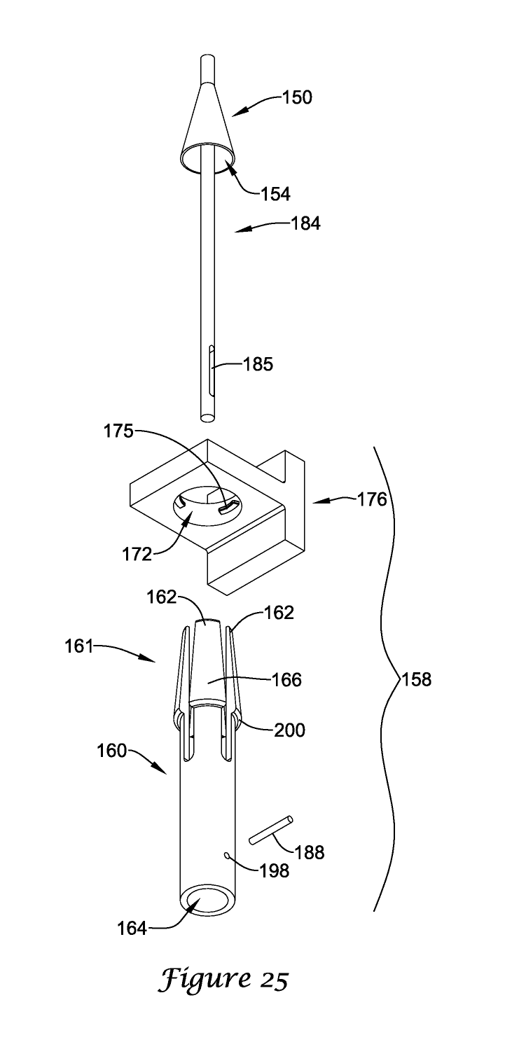

In some embodiments, a medical device system may include at least one actuator element or push-pull rod 184, which may be substantially similar in form and function to the at least one actuator element or push-pull rod 84 discussed above. The at least one actuator element or push-pull rod 184 may include an unlocking member 150 fixedly attached thereto, as seen in FIG. 25, for example. In some embodiments, the unlocking member 150 may include a distally-facing cavity 154 formed therein. In some embodiments, the at least one actuator element or push-pull rod 184 may include an elongate aperture or slot 185 extending transversely therethrough at a distal end thereof.

In some embodiments, the medical device system may include at least one locking element 158. In some embodiments, the at least one locking element 158 may include a first locking portion or post 160 and a second locking portion or buckle 176 configured to receive the first locking portion or post 160. In some embodiments, the first locking portion or post 160 may be fixedly attached to a distal portion of the anchor member or braid 70, similar to the first locking portion or post 60 discussed above. In some embodiments, the second locking portion or buckle 176 may be fixedly attached to a proximal portion of the anchor member or braid 70, similar to the second locking portion or buckle 76 discussed above.

As seen in a partially-exploded view in FIG. 25, the first locking portion or post 160 may include an elongated member having a longitudinally-extending passage 164 extending therethrough and an opening 198 extending transversely therethrough for receiving a pin 188. In some embodiments, the elongated member of the first locking portion or post 160 may include at least two openings 198 positioned on opposing sides of the elongated member and/or the passage 164. In some embodiments, the actuator element or push-pull rod 184 may be slidably disposed within with passage 164 and be releasably coupled to the first locking portion or post 160 by the pin 188 disposed through the opening(s) 198 and the elongated aperture or slot 185 in the distal end of the actuator element or push-pull rod 184. In some embodiments, the first locking portion or post 160 may include one or more apertures or other features provided to aid in attaching the first locking portion or post 160 to the anchor member or braid 70.

In at least some embodiments, the first locking portion or post 160 may include a tapered end portion 161 having a first end and a second end. In some embodiments, the tapered end portion 161 may be configured to engage a circumferential frame of the second locking portion or buckle 176, described in more detail below. In some embodiments, the tapered end portion 161 may be tapered from a first outer extent at the first end to a second outer extent at the second end, wherein the second outer extent is less than the first outer extent when the tapered end portion 161 is in an unstressed or relaxed configuration. In some embodiments, the first end of the tapered end portion 161 may define a shoulder or ridge 200 oriented generally transverse to a longitudinal axis of the elongated member of the first locking portion or post 160. In at least some embodiments, the shoulder or ridge 200 may extend outward from a body of the first locking portion or post 160 and/or may have a greater outer extent than the body of the first locking portion or post 160.

In some embodiments, the tapered end portion 161 may be configured to flex or deflect inwardly as the tapered end portion 161 is translated through a passageway 172 of the second locking portion or buckle 176. In some embodiments, the tapered end portion 161 may include a plurality of legs 162 configured to flex or deflect inwardly toward each other as the tapered end portion 161 is translated through a passageway 172 of the second locking portion or buckle 176. In some embodiments, the tapered end portion 161 may include a tapered surface 166 on each of the plurality of legs 162, the tapered surface 166 extending from the first end to the second end of the tapered end portion 161. In at least some embodiments, the tapered end portion 161 may be disposed at a proximal end of the first locking portion or post 160, as the first locking portion or post 160 is positioned and/or attached to the anchor member or braid 70. In at least some embodiments, the tapered surface(s) 166 may form a wedge-like shape having a narrow end (as viewed transversely or from the side) at the proximal end of the first locking portion or post 160 and a wide end distal of the narrow end. As will be apparent, the tapered surface(s) 166 of the tapered end portion 161 may engaged a distal end of the second locking portion or buckle 176 and urge and/or deflect the tapered end portion 161 inwardly as the first locking portion or post 160 is translated proximally relative to the second locking portion or buckle 176 as a result of proximal retraction of the actuator element or push-pull rod 184.

In at least some embodiments, the second locking portion or buckle 176 of the at least one locking element 158 may include a circumferential frame defining a passageway 172 extending longitudinally therethrough. In some embodiments, the passageway 172 may be configured to receive at least a portion of the elongated member of the first locking portion or post 160 therein. In some embodiments, the circumferential frame may be configured to engage the tapered end portion 161 when the elongated member of the first locking portion or post 160 is translated through the passageway 172. In some embodiments, the second locking portion or buckle 176 may include one or more apertures or other features provided to aid in attaching the second locking portion or buckle 176 to the anchor member or braid 70.

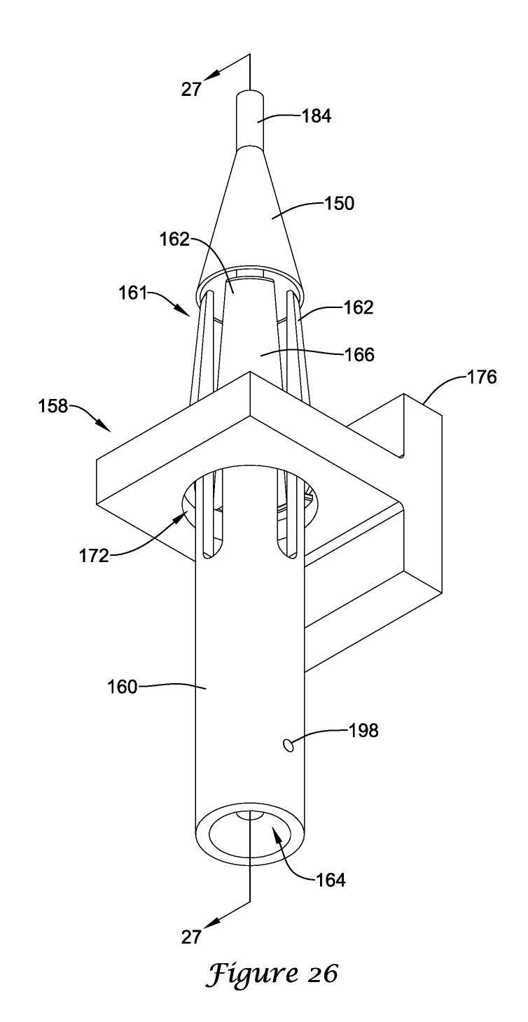

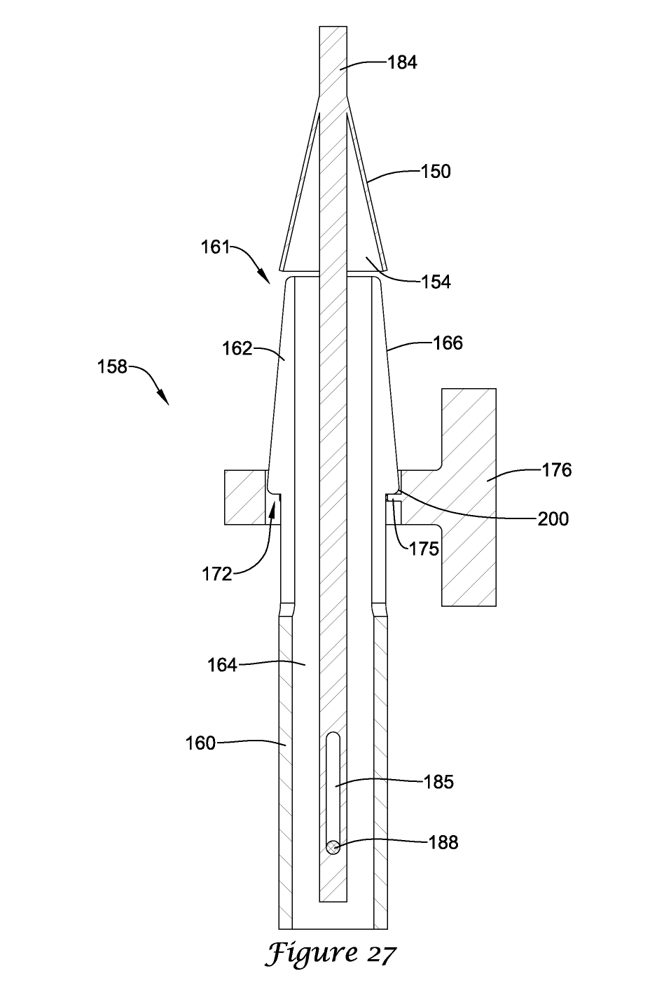

In some embodiments, the circumferential frame of the second locking portion or buckle 176 may include at least one projection 175 extending radially inwardly from the circumferential frame configured to engage the shoulder or ridge 200 of the tapered end portion 161 of the first locking portion or post 160. In some embodiments, the circumferential frame of the second locking portion or buckle 76 may include two, three, four, five, six, or more projections 175 disposed around a perimeter of the passageway 172 of the second locking portion or buckle 76. Additional arrangements are also contemplated. In some embodiments, the at least one projection 175 may extend inward from the circumferential frame into the passageway 172. In some embodiments, translating the second end of the tapered end portion 161 through the passageway 172 of the second locking portion or buckle 176 until the shoulder or ridge 200 has engaged the at least one projection 175 locks the anchor member or braid 70 in the “deployed” configuration, as seen in FIGS. 26 and 27, for example. In some embodiments, the actuator element or push-pull rod 184 extends through the passageway 172 of the second locking portion or buckle 176 and is releasably coupled to the first locking portion or post 160. In at least some embodiments, the actuator element or push-pull rod 184 may be axially translatable through the second locking portion or buckle 176.

FIG. 27 illustrates a partial cross-sectional view of the at least one locking element 158, including the first locking portion or post 160 engaged with and/or locked to the second locking portion or buckle 176. As can be seen in the figure, the legs 162 of the tapered end portion 161 may be disposed within the passageway 172. When the shoulder or ridge 200 engages and/or extends past the at least one projection 175, the anchor member or braid 70 will have assumed the “deployed” configuration, as discussed above.

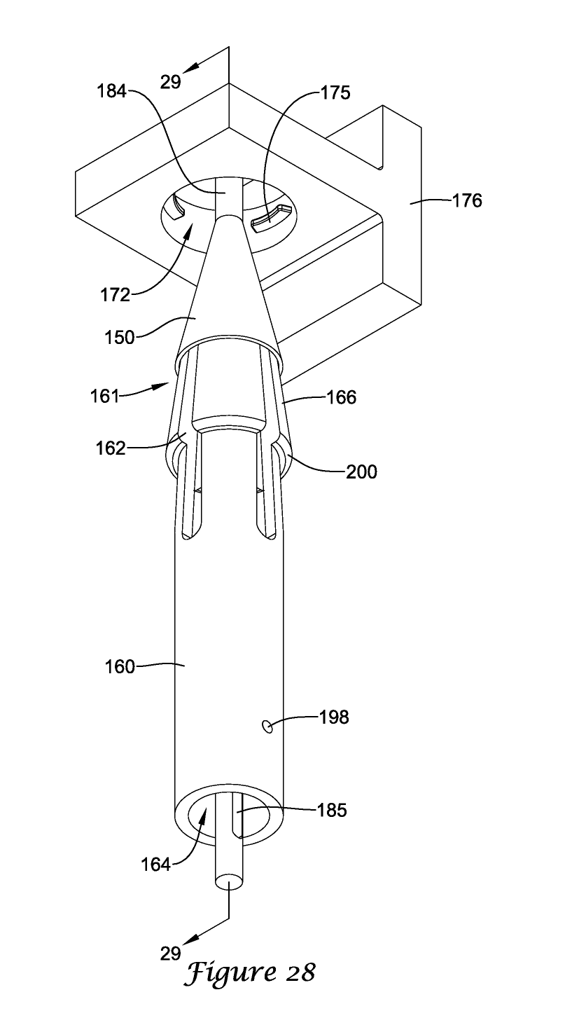

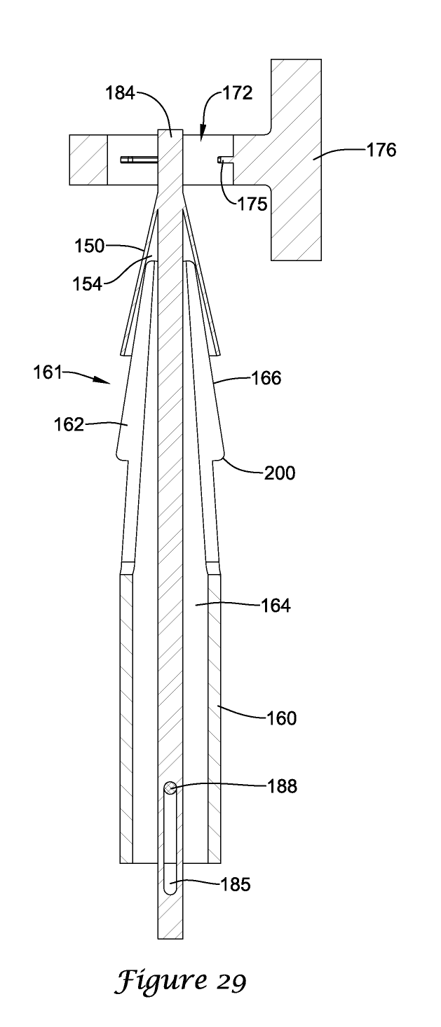

Again, similar to the discussion above, the at least one locking element 158 may be unlocked from the “deployed” configuration by urging the actuator element or push-pull rod 184 distally such that the unlocking member 150 engages the tapered end portion 161 of the first locking portion or post 160 and compresses the legs 162 inwardly toward the actuator element or push-pull rod 184 to disengage the shoulder or ridge 200 from the at least one projection 175 and/or the second locking portion or buckle 176, as seen in FIGS. 28 and 29, for example. Similar to above, the actuator element or push-pull rod 184 and/or the unlocking member 150 may be axially translatable through the second locking portion or buckle 176. In other words, distal axial translation of the actuator element or push-pull rod 184 may compresses the first locking portion or post 160 of the at least one locking element 158 laterally inward, thereby disengaging the first locking portion or post 160 from the second locking portion or buckle 176 of the at last one locking element 158 fixedly attached to the anchor member or braid 70. When the clinician is satisfied with the placement of the medical implant, the pin(s) 188 may be pulled to decouple and/or disengage the actuator element or push-pull rod 184 from the first locking portion or post 160.

The materials that can be used for the various components of the medical device system 10 (and/or other systems disclosed herein) and the various tubular members disclosed herein may include those commonly associated with medical devices. For simplicity purposes, the following discussion makes reference to the outer sheath 12 and/or the inner catheter 14. However, this is not intended to limit the devices and methods described herein, as the discussion may be applied to other similar tubular members and/or components of tubular members or devices disclosed herein, such as, but not limited to, the actuator element or push-pull rod, the unlocking member, the first locking portion, the second locking portion, and/or elements or components thereof. In some embodiments, the outer sheath 12 and/or the inner catheter 14 may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of the outer sheath 12 and/or the inner catheter 14 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the medical device system 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the medical device system 10 to achieve the same result.

In some embodiments, a degree of Magnetic Resonance Imaging (MRI) compatibility is imparted into the medical device system 10. For example, the outer sheath 12 and the inner catheter 14, or portions thereof, may be made of a material that does not substantially distort the image and create substantial artifacts (i.e., gaps in the image). Certain ferromagnetic materials, for example, may not be suitable because they may create artifacts in an MRI image. The outer sheath 12 and the inner catheter 14, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

A sheath or covering (not shown) may be disposed over portions or all of the outer sheath 12 and the inner catheter 14 that may define a generally smooth outer surface for the medical device system 10. In other embodiments, however, such a sheath or covering may be absent from a portion of all of the medical device system 10, such that the outer sheath 12 and the inner catheter 14 may form an outer surface. The sheath may be made from a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the medical device system 10 (including, for example, the exterior surface of the outer sheath 12 and the inner catheter 14) may be sandblasted, beadblasted, sodium bicarbonate-blasted, electropolished, etc. In these as well as in some other embodiments, a coating, for example a lubricious, a hydrophilic, a protective, or other type of coating may be applied over portions or all of the sheath, or in embodiments without a sheath over portion of the outer sheath 12 and the inner catheter 14, or other portions of the medical device system 10. Alternatively, the sheath may comprise a lubricious, hydrophilic, protective, or other type of coating. Hydrophobic coatings such as fluoropolymers provide a dry lubricity which improves device handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found, for example, in U.S. Pat. Nos. 6,139,510 and 5,772,609, which are incorporated herein by reference.

The coating and/or sheath may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present invention.

The following documents are herein incorporated by reference in their entirety:

U.S. Patent Application Publication No. US 2007/0112355;

U.S. Patent Application Publication No. US 2010/0219092;

U.S. Patent Application Publication No. US 2010/0280495;

U.S. Patent Application Publication No. US 2011/0257735;

U.S. Patent Application Publication No. US 2013/0123796; and

U.S. Patent Application Publication No. US 2013/0158656.

It should be understood that this disclosure is, in many respects, only illustrative. Changes may be made in details, particularly in matters of shape, size, and arrangement of steps without exceeding the scope of the invention. This may include, to the extent that it is appropriate, the use of any of the features of one example embodiment being used in other embodiments. The invention's scope is, of course, defined in the language in which the appended claims are expressed.

What is claimed is:

1. A medical device, comprising:
an elongate delivery sheath;
a valve replacement implant disposed within a lumen of the delivery sheath, the implant including an anchor member reversibly actuatable between a delivery configuration and a deployed configuration;
wherein the implant includes at least one locking element configured to lock the anchor member in the deployed configuration; and
at least one actuator element configured to engage the at least one locking element and actuate the anchor member between the delivery configuration and the deployed configuration;
wherein the at least one actuator element includes an unlocking member configured to compress a first locking portion of the at least one locking element, thereby unlocking the anchor member from the deployed configuration.

2. The medical device of claim 1, wherein the first locking portion of the at least one locking element is fixedly attached to the anchor member and a second locking portion of the at least one locking element is fixedly attached to the anchor member, the first locking portion and the second locking portion being longitudinally movable relative to each other in the delivery configuration.

3. The medical device of claim 2, wherein the first locking portion is fixedly attached to a distal portion of the anchor member.

4. The medical device of claim 2, wherein the second locking portion is fixedly attached to a proximal portion of the anchor member.

5. The medical device of claim 2, wherein the first locking portion engages the second locking portion in the deployed configuration.

6. The medical device of claim 5, wherein the second locking portion includes a circumferential frame defining a passageway and the first locking portion includes an elongated member having a passage therethrough;
wherein the passageway is configured to receive the elongated member therein.

7. The medical device of claim 6, wherein the tubular member includes a tapered end portion configured to engage the circumferential frame when the elongated member is translated through the passageway.

8. The medical device of claim 7, wherein the tapered end portion includes a first end and a second end, the tapered end portion being tapered from a first outer extent at the first end to a second outer extent at the second end;
wherein the second outer extent is less than the first outer extent, and the first end defines a shoulder of the elongated member oriented generally transverse to a longitudinal axis of the elongated member.

9. The medical device of claim 8, wherein the tapered end portion is configured to flex inward as the tapered end portion is translated through the passageway.

10. The medical device of claim 8, wherein the circumferential frame includes at least one recess extending outward from the passageway, the at least one recess being configured to engage the shoulder.

11. The medical device of claim 10, wherein translating the second end of the tapered end portion through the passageway until the shoulder has engaged the at least one recess locks the anchor member in the deployed configuration.

12. The medical device of claim 8, wherein the circumferential frame includes at least one projection extending inwardly into the passageway, the at least one projection being configured to engage the shoulder.

13. The medical device of claim 8, wherein the at least one actuator element extends through the second locking portion and is releasably coupled to the first locking portion, the at least one actuator element being axially translatable through the second locking portion.

14. The medical device of claim 13, wherein the unlocking member is axially translatable through the second locking portion.

15. The medical device of claim 14, wherein the unlocking member includes a cavity disposed therein, the cavity being configured to receive at least a portion of the first locking portion.

16. The medical device of claim 13, wherein axial translation of the at least one actuator element in a first direction actuates the anchor member from the delivery configuration to the deployed configuration.

17. The medical device of claim 16, wherein axial translation of the at least one actuator element in a second direction opposite from the first direction engages the unlocking member with the tapered end portion to unlock the anchor member from the deployed configuration.

18. The medical device of claim 17, wherein further axial translation of the at least one actuator element in the second direction actuates the anchor member from the deployed configuration to the delivery configuration.

19. A medical device, comprising:
an elongate delivery sheath;
a medical implant disposed within a lumen of the delivery sheath and attached to an inner catheter, the implant including an anchor member reversibly actuatable between an elongated delivery configuration and an expanded deployed configuration;
wherein the implant includes first, second, and third locking elements configured to lock the anchor member in the deployed configuration; and
first, second, and third actuator elements configured to engage the first, second, and third locking elements and actuate the anchor member between the delivery configuration and the deployed configuration;
wherein the first, second, and third actuator elements each include an unlocking member having a cavity configured to receive and compress a first locking portion of each of the first, second, and third locking elements to unlock the anchor member from the deployed configuration.

20. The medical device of claim 19, wherein distal axial translation of the first, second, and third actuator elements compresses the first locking portion of each of the first, second, and third locking elements laterally inward, thereby disengaging the first locking portion from a second locking portion of the locking elements fixedly attached to the anchor member.
